(19) United States
(12) Patent Application Publication
(10) Pub. No.: US 2019/0024114 A1
(43) Pub. Date: Jan. 24, 2019

(54) METHOD OF PRODUCING A PRODUCTION CELL LINE

(71) Applicant: Anton Bauer, Kirchberg/Wagram (AT)

(72) Inventors: Anton Bauer, Kirchberg/Wagram (AT); Gottfried Himmler, Gross-Enzersdorf (AT)

(21) Appl. No.: 16/069,164

(22) PCT Filed: Jan. 16, 2017
(86) PCT No.: PCT/EP2017/050793

§ 371 Date: Jul. 10, 2018

(30) Foreign Application Priority Data
Jan. 14, 2016 (EP) 16151178.7

(51) Int. Cl. C12N 15/85

(52) U.S. Cl. CPC C12N 15/85

(57) ABSTRACT

A method for producing a eukaryotic production cell line expressing a protein of interest (POI), comprising a) incorporating a gene of interest (GOI) encoding said POI into the chromosome of a eukaryotic host cell within an exogenous euchromatin protein expression locus by transfection, thereby obtaining a repertoire of recombinant host cells in a pool; b) selecting a single cell from said pool within 12 days after transfection, wherein selecting is at least according to the expression of said GOI or a marker indicating said expression; and c) isolating and expanding the selected single cell, thereby obtaining the production cell line.

The invention relates to a method for producing a eukaryotic production cell line expressing a protein of interest (POI).

### BACKGROUND

Efficient and high yield production of recombinant proteins for therapeutic or other commercial use requires stable, highly expressing recombinant cell lines. Eukaryotic cells engineered to express the desired protein at high titers in a bioreactor are typically employed in the manufacturing process of such biopharmaceuticals. For this purpose, eukaryotic cell lines are transfected with an expression vector containing the gene encoding the desired protein. A suitable single cell clone has then to be identified and selected. This step is crucial for the generation of cell lines capable of stable, reliable and reproducibly expressing high yields of desired protein (Wurm, F. M. Nature Biotechnology 22, 1393-1398 (2004)). Current methods for the identification and selection of a cell clone with optimal production and growth profile are time-consuming and laborious, involving screening of numerous transfected cells.

Most of the currently used methods utilize the ability of an additional gene product included in the recombinant DNA containing the gene-of-interest (GOI), to provide for a selective advantage for the transfected cell over the non-transfected cell, for example resistance to an antibiotic or ability to grow in a selective medium (e.g., Zboray et al., Nucleic Acid Research 43 (16), 1-14 (2015)). Zboray et al. employed a bacterial artificial chromosome vector that is stably integrated into the host cell chromosome. Clonal protein production was directly proportional to integrated vector copy numbers and remained stable during 10 weeks without selection pressure. Single cell clones were obtained by limiting dilution technique. Blaas et al. also describe bacterial artificial chromosomes to improve recombinant protein production in mammalian cells (Blaas et al. BMC Biotechnology 2009, 9:3). Again, single cell clones were established using a dilution technique.

WO2010060844A1 discloses a bacterial chromosome vector used to engineer a host cell for recombinant protein production, employing a Rosa26 locus which contains regulatory elements for open chromatin formation and an expression chromatin structure.

Selection methods based on antibiotic resistance generally use antibiotic concentrations that are rather mild to avoid any indirect toxicity to transfected cells. As a result, transfected cultures are maintained under constant presence of antibiotics until the entire non-transfected part of the transfection cell population is removed from the culture while still maintaining viability over 50% of the total population at all times.

Transient expression of non-integrated DNA in first weeks of culture is contributing to a lengthy protocol for selection of stable cell lines.

In some strategies, the antibiotics concentration is gradually increased during the selection phase. This cultivation period under selective conditions uses significant resources and time, generally taking about a month from transfection until generation of a stable pool of cells. Furthermore, selection pressure over a prolonged period of time increases the probability for further chromosomal changes or changes in the expression pattern of the host cell and cellular stress.

Once the stable pool is generated, limiting dilution is setup to isolate single clones. Cells are diluted and seeded in 96-well or 384-well plates to start with a single cell that can expand. A main disadvantage of this technique is that certain clones, which may not be best producers, could divide faster and as a result the best producer is diluted out from the culture. Therefore, to isolate a “high producer” clone by limiting dilution requires established detection methods as well as tedious and careful screening of a high number of clones to identify the best producers in a selected pool.

The introduction of green fluorescent protein and other fluorescent proteins developed therefrom allowed identification of transfected cells based on co-expression of the desired recombinant protein with the fluorescent protein. In particular, flow cytometry methods (e.g. FACS) have been employed for the rapid identification and isolation of production clones from a heterogeneous population of transfected cells involving the selection of a fluorescent co-marker, e.g. GFP, or staining of cells with fluorescent labels detecting a marker protein on the cell membrane of the host cell. The drawback of this approach is that expression of the desired protein may actually be compromised due to high expression of the fluorescent marker, and the ultimate yield of the desired protein may thus be reduced. Furthermore, selection is primarily based on high levels of the fluorescent marker which does not always correlate with high expression of the desired protein.

DeMaria et al. (Biotechnol Prog 2007, 23, 465-472) describe a selection method based on flow cytometry using expression of a cell surface protein not normally expressed in the host cell as a reporter protein. The genes encoding the reporter protein and the protein of interest are linked by an IRES, enabling their transcription in the same mRNA, and expression of the reporter protein is detected with a fluorescently labeled antibody.

As an alternative approach to using a reporter gene which is either directly or indirectly labelled, methods have been developed based on detection of the desired protein. For example, US2013009259 describes a FACS approach for single cell sorting, selecting high production clones through direct labeling of the desired protein on the cell membrane. After selection of a clone based on its fluorescence intensity, further subcloning steps are required to ensure the genetic stability of the selected clone and ability to produce the desired protein reproducibly over several generations.

Okumura et al. (Journal of Bioscience and Bioengineering 120 (3) 340-346 (2015)) report an enrichment strategy for high-producing cells employing flow cytometry. In this study, eukaryotic cells were transfected with an expression vector for a monoclonal antibody, resulting in a pool of cells with a huge variety of monoclonal antibody expression levels. Cells in this pool were stained with a fluorescent-labeled antibody binding to the mAb present on the cell surface during secretion and sorted by flow cytometry, setting cell size and intracellular density gates based on forward light scatter (FSC) and side light scatter (SSC), thereby preselecting cell fractions based on their FSC and SSC gates. These preselected cell fractions were then sorted by further flow cytometry analysis based on fluorescence levels.

FSC and SSC gating was also employed by Shi et al. to select live cells which are further screened and sorted based on fluorescence intensity (Journal of Visualized Experiments (55), e3010:1-5).

Label free cell separation and sorting in microfluidic systems is described by Gossett et al. (Anal Bioanal Chem 2010, 397:3249-3267).

WO2010128032A1 discloses CHO cell lines comprising vector constructs comprising a certain expression cassette to overexpress a mutant of the ceramide transfer protein (CERT), namely CERT S132A to enhance its secretion capabilities. Cell lines are selected for an increased level of CERT expression by single cell sorting.

US2010021911A1 discloses production host cell lines comprising vector constructs. Whereas a first vector construct comprises a DHFR expression cassette, a second vector construct comprises a gene of interest and a selection and/or amplification marker other than DHFR.

EP2700713A1 discloses a screening and enrichment system for protein expression in eukaryotic cells using a tricistronic expression cassette. Cells expressing high levels of a protein of interest are screened, sorted and/or enriched by means of a reporter protein.

WO2015092735A1 discloses eukaryotic cells expressing a protein of interest, wherein the effect of the expression product of an endogenous gene C12orf35 is impaired in said cell.

WO2012085911A1 discloses membrane-bound reporter molecules and their use in cell sorting.

WO2008145133A2 discloses a method for manufacturing a recombinant polyclonal protein composition, wherein a collection of cells transfected with a collection of variant nucleic acids sequences is transfected and further cultured for expression of the polyclonal protein.

Current methods using flow cytometry require several weeks after transfection for gene amplification and/or generation of a stable pool of cells, which can then be screened. In addition, selected clones need to be re-cloned, and further cultivated to finally identify the most suitable clone for stable high yield production.

### SUMMARY OF THE INVENTION

It is an object of the invention to provide a simple and fast method to generate, identify and select a single cell which qualifies as a first cell of a stable production cell line capable of producing a POI with high yield.

The object is solved by the subject matter as claimed.

According to the invention, there is provided a method for producing a eukaryotic production cell line expressing a protein of interest (POI), comprising

a) incorporating a gene of interest (GOI) encoding said POI into the chromosome of a eukaryotic host cell within an exogenous euchromatin protein expression locus by transfection, thereby obtaining a repertoire of recombinant host cells in a pool;

b) selecting a single cell from said pool within 12 days after transfection, wherein selecting is at least according to the expression of said GOI or a marker indicating said expression; and

c) isolating and expanding the selected single cell, thereby obtaining the production cell line.

Specifically, a selection marker gene is additionally incorporated into the host cell and the repertoire of recombinant host cells is maintained in said pool under corresponding selection pressure conditions, and wherein said selecting is at least according to any of the transfected marker gene, the marker, or the function of said marker. According to a specific embodiment, the pool is kept within a containment under said selection pressure for only a short period of time before single cell sorting, e.g. no longer than 12 days after transfection, preferably no longer than any one of 11 days, 10 days, 9 days, 8 days, 7 days, 6 days, 5 days, 3 days, 2 days, or 1 day.

The selection marker specifically provides the cell with a survival and/or growth advantage when maintained or cultivated under corresponding selective conditions, herein also referred to as “selection pressure” or “selective pressure” that allows differentiation between the robust cells and non-robust or dead cells. It is specifically preferred to employ the selection step b) directly from the pool, without any pre-selection. Thus, the repertoire can be directly undergoing single cell sorting without pre-screening under selection pressure.

In some embodiments, isolating and expanding the selected single cell according to step c) of the methods described herein follows immediately step b) without any further limited dilution step. In some embodiments, selecting a single cell according to step b) of the methods described herein immediately follows step a), preferably within a maximum of any one of 7 days, 6 days, 5 days, 3 days, 2 days, or 1 day, after step a). Specifically, said single cell sorting immediately follows the transfection of said host cell to incorporate the GOI without any cell division, or in the first or second generation, or within 5 or 10 or maximally 15 generations.

In some embodiments, selecting a single cell according to step b) of the methods described herein is by sorting according to at least one intrinsic physical biomarker only, preferably in a single step procedure, optionally followed by further sorting based on productivity.

Specifically, selecting a single cell from a repertoire of recombinant host cells according to the methods described herein is by cell sorting without using a fluorescent label, preferably without using any label.

Thus, according to a preferred embodiment, the production clone can be produced from a single cell as described herein, directly upon stably integrating the GOI into the host cell, followed by the single cell sorting, within a short timeframe.

Specifically, the selected single cell is a recombinant host cell which is immediately ready for expanding to a production host cell line without further cell engineering and/or optimization steps and/or selection pressure. According to a specific aspect, the GOI is stably integrated in the host cell chromosome, preferably within an expression construct within or comprising an expression locus or at least part of an expression locus, thereby providing the operable euchromatin protein expression locus within the host cell chromosome.

Hereinafter, the term “expression construct” is used which can be any of the expression cassettes, expression loci, or vectors, as further described herein.

Specifically, said exogenous euchromatin protein expression locus is integrated into the host cell via a vector comprising said locus, preferably an artificial chromosome vector, such as any one of a bacterial artificial chromosome (BAC), a P1-derived artificial chromosome (PAC), a yeast artificial chromosome (YAC), human artificial chromosome (HAC), or a cosmid. Such vectors can be incorporated into the host cell genome by a technique suitable for transfecting the host cell.

Specifically, said expression construct is an artificial chromosome vector, preferably any one of a BAC, PAC, YAC, HAC, or a cosmid. Specifically, the expression construct is either circular or first linearized followed by transfection of the host cell to enable chromosomal integration of one or more linearized expression cassettes.

According to a specific example, the BAC comprising the locus Rosa26, Rosa26 BAC (Rosa26 locus corresponding to clone RPCI-24-85L15 (ID:760448); GRCm38.p3 C57BL/6J: Chr. 6 (NC_000072.6): 112, 952, 746-113, 158, 583; source: NCBI; SEQ ID NO:1) is used, specifically to transfect mammalian host cells thereby producing recombinant host cells, e.g. hamster cells such as CHO. Further preferred BAC vectors are e.g., BAC comprising the locus Rps21, Rps21 BAC (Rps21 locus corresponding to clone RP23-88D12 (ID:627270;), SEQ ID NO:2), BAC including locus Actb, Actb BAC (Actb locus corresponding to clone RP23-5J14 (ID:601738;), SEQ ID NO:3) and BAC including locus Hprt, Hprt BAC (Hprt locus corresponding to clone RP23-412J16 (ID:732121;), SEQ ID NO:4), (BAC-PAC Resources: Children's Hospital Oakland Research Institute (CHORI)).

In some embodiments, said vector is integrated randomly into the chromosome of the host cell or by site-specific integration. Specifically, said GOI is randomly incorporated into the euchromatin protein expression locus, or by site-specific integration. Specifically, the GOI is incorporated into the locus within an operable expression cassette.

Specifically, an expression construct can be used which is an artificial chromosome vector that is randomly incorporated into the chromosome of the host cell according to the methods described herein. In some embodiments, said expression construct is an artificial chromosome which is incorporated into the chromosome of the host cell by site directed integration (e.g. homologous recombination or targeted gene integration into site-specific loci e.g., using CRISPR/Cas9 genome editing system). In some embodiments, the expression construct is a plasmid, which is stably incorporated into the chromosome of the host cell by site directed integration (e.g. homologous recombination or targeted gene integration into site-specific loci e.g., using CRISPR/Cas9 genome editing system).

According to a specific embodiment, one or more copies of the GOI are incorporated into the host cell chromosome, preferably at least or more than 5 copies, or at least 10, or at least 15, or at least 20 copies of the GOI. This can e.g. be achieved by the selected amount of GOI DNA used for host cell transfection. According to a specific embodiment, the selected single cell is characterized by a GOI copy number of at least or more than 5 copies, or at least 10, or at least 15, or at least 20 copies of the GOI.

According to a specific embodiment, said expression construct comprises one or more copies of the GOI and is used to transfect the host cell, thereby incorporating or establishing one or more euchromatin protein expression loci within the chromosome of the host cell which comprise one or more copies of the GOI each.

According to a further specific embodiment, said expression construct can be used to first transfect the host cell without the GOI, thereby preparing the host cell by incorporating or establishing one or more euchromatin protein expression loci within the chromosome of the host cell. In a second step, one or more copies of the GOI can be incorporated into a euchromatin protein expression locus of the host cell chromosome.

Specifically, said locus is exogenous and heterologous to the host cell.

According to a specific aspect, any exogenous locus may be used which is characterized by the open chromatin structure of a euchromatin protein expression locus. Such loci are typically understood to be constitutively active as expression locus, e.g. any of the Rosa26, Rps21, Actb, or Hprt, or any locus of a housekeeping gene, which is heterologous or foreign to the host cell.

According to a further specific aspect, any exogenous locus may be used, which is characterized by the open chromatin structure of a euchromatin protein expression locus. The exogenous locus (sometimes referred to as heterologous) is typically, but not necessarily, artificial or non-naturally occurring within the host cell chromosome, and specifically obtained from a source other than the host cell, such as from a different cell type or species. Yet, it is specifically preferred that both, the locus and the host cell is of mammalian or avian origin.

One or more copies of the expression construct may be integrated into the chromosome, preferably at least 1, 2, 3, 4, 5, 6, 7, 8, 9, or 10 copies of the expression construct, or even more than 10 copies, specifically, at least 15, 20, 25, 30, 35, 40, 45, 50 or even at least 60, 70, 80, 90, or 100 copies. The expression constructs may be integrated at one or more chromosomal loci, e.g. following transfection of the host cell line with the circular or linearized expression construct.

Bacterial artificial chromosome vectors and other vectors carrying enough DNA elements to shield against adverse neighboring chromatin effects can integrate anywhere in the host cell chromosome and support expression of genes encoded on the vector. In some embodiments, the integration may be at a chromosomal locus of a gene which is abundantly expressed by the host cell.

The repertoire of recombinant host cells specifically contains a pool of clones which are characterized by the stable integration of the expression construct into the host cell chromosome. The selecting step may immediately follow the incorporation step without previous propagation and/or enrichment of the high-producer cell lines. In some embodiments, selecting a single cell according to step b) of the methods described herein follows step a) immediately, preferably within a maximum of any one of 12 days, 11 days, 10 days, 9 days, 8 days, 7 days, 6 days, 5 days, 3 days, 2 days, or 1 day, after method step a) of the method described herein, or the transfection.

According to a specific embodiment, a pre-selection may be performed, e.g. to deplete non-functional clones, e.g. which do not survive a selective pressure, or where the chromosomal incorporation of the expression construct was not successful (e.g. removing impaired or dead cells, negative selection). Any pre-selection of cells from the pool (before single cell selection) is preferably carried out after the transfection according to step a) and before or during single cell sorting, yet, not extending the time to selecting the single cell after transfection, e.g. within 12 days after transfection.

According to a further specific embodiment, a further selection step may be performed, e.g. to enrich those clones which are characterized by a high copy number of the expression construct and/or a high copy number of the GOI (e.g. selecting according to the expression of a selection marker or according to the yield of POI production, positive selection). Such selection is preferably carried out after the single cell sorting. The transfected clones can also be enriched for clones containing a high copy number of the expression construct or GOI to yield a positively selected fraction of clones, which likely includes the high-producers. Thus, the likelihood of selecting a single cell with the potential of a high productivity of POI expression can be increased by such enrichment. Optionally, the method may comprise a further step of selection or enrichment of a cell population, e.g. including a viability enrichment step, a chromatographic enrichment step or an assay enrichment step.

Specifically, the method as described herein further comprises incorporating a selection marker gene, e.g. employing an expression construct which further comprises a selection marker gene, for coexpression of a selection marker with the POI. The selection marker may be engineered into the expression construct, such as to enable selection of clones which have incorporated the expression construct including the marker gene. Alternatively, the selection marker may be incorporated into the expression construct and/or the host cell chromosome only as an inactive gene, and becomes active and detectable upon successful chromosomal integration. Thus, the selection marker can be used as a qualitative read-out, indicating the successful transfer of the gene in the repertoire of recombinant host cells.

According to a specific aspect, one or more copies of the selection marker can be integrated into the host cell chromosome together with and near to the GOI. Specifically, the number of selection marker genes and the level of expressed selection marker can be indicative of the productivity of the recombinant host cell. Accordingly, the selection marker may be used as a quantitative indicator of POI expression. In particular, the selection marker may indicate the successfully integrated and/or functional copy number of the expression construct and/or the GOI. According to a specific aspect, the selection marker gene is operably linked to a GOI, thereby obtaining a level of expressed selection marker indicative of the level of expressed POI. In some embodiments, the gene copy number of the GOI directly correlates with the specific productivity for the POI, and the selection marker gene is integrated together with the GOI in the expression vector at a fixed ratio. In some embodiments, the copy number of the selection marker gene as well as its expression level and consequently its activity directly correlate with the POI expression level.

The pre-selection is commonly performed upon detecting the marker directly or by indirect means. The positive pre-selection method, e.g. the presence of a viability or resistance marker, may also include a maintenance or culturing step, in which the repertoire of recombinant host cells can be maintained or cultured with suitable medium under selective pressure, e.g. under conditions that favor the survival of robust clones, or clones which are characterized by the stable integration of the expression construct and optionally which reflect the copy number of the integrated expression construct or the copy number of the GOI. In some embodiments, the repertoire of cells is maintained or cultured under these conditions in one or more stages, e.g. with a high selective pressure, such as for up to 12 days, e.g. for a maximum of any one of 12 days, 11 days, 10 days, 9 days, 8 days, 7 days, 6 days, 5 days, 3 days, 2 days, or 1 day. Alternatively, more than one stage with increasing selective pressure may be applied, e.g. each for at least 1 day, or at least 2, 3, 4, 5, 6, 7, 8, or 9 days, e.g. up to 12 days.

In some embodiments, the repertoire of cells is selected for the single cell as described herein within for at most any one of 7 days, 6 days, 5 days, 3 days, 2 days, or 1 day, in particular wherein no specific cultivation step is carried out and the selection is e.g. immediately following after the transfection under the selective pressure, optionally employing a pre-selection of robust cells using selective pressure or high selective pressure as further defined herein.

Specifically, before selecting the single cell, said repertoire of recombinant cells is grown to coexpress said POI and said selection marker under high selective and stringent conditions, and a fraction of resistant (herein also referred to as “robust”) cells is pre-selected.

According to a specific embodiment, said selection marker gene is an antibiotic resistance marker gene or a metabolic function selection marker gene, which co-expresses a selection marker with the POI.

According to a specific embodiment,
a) said selection marker gene is an antibiotic resistance marker gene or a metabolic function selection marker gene; and
b) before selecting the single cell, said repertoire of recombinant cells is grown to coexpress said POI and said selection marker under selective conditions or high selective condition, and a fraction of resistant cells is pre-selected.

Specifically, the selection marker gene is
a) a metabolic function marker gene, preferably a gene encoding any of ADA, DHFR, GS, histidinol D, TK, XGPRT, or CDA; or
b) an antibiotic resistance marker gene, preferably a gene conferring resistance to any of
   i. aminoglycosides, preferably any of neomycin (G418), geneticin, kanamycin, streptomycin, gentamicin, tobramycin, neomycin B (framycetin), sisomicin, amikacin, isepamicin or hygromycin B;
   ii. puromycin;
   iii. bleomycines, preferably any of bleomycin, phleomycin, or zeocin;
   iv. blasticidin; or
   v. mycophenolic acid.

Specifically, the selection marker gene and the GOI are both incorporated into the expression construct at a defined ratio. In particular, the ratio may be predefined, e.g. by engineering an expression cassette or expression construct containing both, the selection marker gene and a predefined number of one or more copies of the GOI. According to a specific example, equal numbers of the selection marker gene and the GOI are incorporated into the expression cassette or the expression construct, referred to as 1:1 ratio. Alternatively, the predefined ratio may be less than 1:1, e.g. 1:2 (indicating 1 selection marker gene per 2 copies of GOI), or 1:3, or 1:4, or 1:5, or even less. The GOI copy number may be increased by using a defined amount of GOI for transfection, or by precise integration of the number of genes into the expression construct, e.g. by means of a specific number of expression cassettes, or by gene stacking. For example, genes may be repeatedly added, e.g. by tandem repeats, into a site within an expression construct or into a chosen locus of the host cell chromosome, in a precise manner. In addition, method steps of removing any additional foreign DNA elements such as selectable marker genes are provided to reduce the defined ratio of marker genes to GOI.

Specifically, said expression construct is randomly incorporated into the chromosome of the recombinant cell, or by site-specific integration. Upon random integration, the repertoire of recombinant cells may be pre-selected for the expression rate, indicating the chromosomal locus of high translational or expression activity, e.g. the locus brought along by the expression vector as in the case of e.g. a BAC expression vector, or of a chromosomal locus of an abundant protein or a “hot-spot”. The “hot-spot” means a position in the chromosome of a host cell which provides for a stable and highly expressionally-active, preferably transcriptionally-active, production of a product. The hot-spot is typically characterized by the open chromatin structure. The euchromatin protein expression locus as described herein is a specific example of a hot spot, if operable to express a gene contained within the locus.

Random integration is typically by non-homologous recombination, thus, without the need to construct matching (homologous) sequences for recombining the 5′ and 3′ terminal sequences of the expression construct with the endogenous target chromosomal sequence.

The site-specific integration may be performed by using an expression construct in conjunction with an insert that recognizes the target site of integration, e.g. employing site-specific DNA recombinase. In particular, an exogenous expression construct can be integrated into an endogeneous recombination target site, such as a wild-type or mutant FRT site or a lox site. In case the recombination target site is a FRT site, the host cells need the presence and expression of FLP (FLP recombinase) in order to achieve a cross-over or recombination event. In case the recombination target site is a lox site, the host cells needs the presence and expression of the Cre recombinase. Specifically, the site-directed integration can be obtained by a site-directed recombination-mediated cassette exchange. Typically, the integration of the expression construct in a site-directed way is by homologous recombination of matching sequences.

Specifically, the method step a) of the method described herein comprises incorporating said GOI into said locus by site-specific integration.

Specifically, said host cell is a mammalian, in particular human, hamster, mouse, monkey, dog, or avian host cell, preferably any one of HEK293, VERO, HeLa, Per.C6, HuNS1, U266, RPMI7932, CHO, BHK, V79, COS-7, MDCK, NIH3T3, NS0, SP2/0, or EB66 cell, any derivatives and/or progeny thereof. Specifically, production cell lines commonly used for pilot scale or industrial scale protein or metabolite production may serve as a host cell for the purpose described herein. Exemplary host cells are BHK, BHK21, BHK-TK⁻, CHO, CHO-DG44, CHO-DUXB11, CHO-DUKX, CHODUKX B11, CHO-K1, CHO Pro-5, CHOK1SV, CHO/CERT2.20, CHO/CERT2.41, CHO-S, V79, B14AF28-G3, COS-7, U266, HuNS1, CHL, HeLa, HEK293, MDCK, NIH3T3, NS0, PER.C6, SP2/0, VERO or EB66 cell.

According to a specific example, the locus is a murine Rosa26 locus, e.g. as used in the Examples described herein, or a mammalian homolog thereof. Specifically, such locus is used for engineering a CHO production host cell and respective cell line.

Specifically, said repertoire of recombinant host cells covers host cells which differ in at least one of
a) the copy number of said GOI;
b) the chromosomal locus or chromosomal loci where the GOI is incorporated;
c) the genetic stability, or
d) the epigenetic stability.

Upon stable chromosomal integration of the expression construct, the genetic stability should be principally high, but may still vary because of morphological changes of the cell. It turned out that cell intrinsic parameters and particularly the physical appearance of the cell can change indicating genetic and/or epigenetic instability. Thus, stable producer cells can be sorted according to such cell intrinsic parameters. Genetic stability and epigenetic stability of the expression locus of particular importance to produce a master cell bank and working cell lines of the production host cell, such as to reproducibly use a production host cell line. The cell line with genetic and epigenetic stability maintains the genetic properties over a prolonged period of time and can be used in a prolonged production phase, e.g. effectively producing the POI, at a high expression level, e.g. at least at a μg level (μ per mL), even after about 10 or 20 generations in the cell culture, preferably at least 30 generations, more preferably at least 40 generations, most preferred of at least 50 or 70 generations. Genetic and epigenetic stability of the expression locus of the cell line is a great advantage when used for industrial scale protein production. The genetic and the epigenetic stability of the expression locus confer that the transcription levels for mRNA encoding the POI and for mRNA encoding the marker protein are not significantly altered (e.g. less than +/−50%, or 40%, or 30%, or 20%, or 10% variance) comparing their levels during the first 10 or 20 generations with their levels after 20 or 40 or 70 generations.

Specifically, said selecting of a single cell from the pool is further by determining any one or more of intrinsic physical biomarkers. Specifically, said selection is according to any of or at least one of cell size, cell cytoplasmic granularity, polarizability, refractive index, or cell membrane potential. Any of such intrinsic biomarkers is determined based on the shape, morphology, appearance and/or function of the cell, which is independent from the POI production. Any transfected cell which is negatively selected because of deformed or deviant intrinsic physical parameters is considered not suitable for the purpose of producing a production cell line. Any transformant cell which is positively selected because it complies to the predefined parameters indicative of the intrinsic physical characteristics, is sorted to further proceed with the manufacture of the production cell line.

According to a specific embodiment, said selecting (also referred to as sorting) is by a single cell sorting technique employing an optical flow cytometry method, preferably using forward light scatter (FSC) and/or side light scatter (SSC), or a microfluidic systems such as droplet based microfluidics or Raman-activated cell sorting or applying acoustic radiation force—according to physical differences in the properties of cells including size, shape, volume, density, elasticity, hydrodynamic property, polarizability, light scattering, dielectrophoresis, and magnetic susceptibility. Such methods provide for the sorting and isolation of single cells in the clonal population by measuring the predefined selection parameter indicative of the intrinsic physical biomarker or respective cell characteristics. For example, the cells are sorted by identifying cells having a specific phenotype, e.g., viability, size, morphology, permeability, density, etc. In one embodiment, cells may be sorted in one or more stages, e.g. upon a first sorting step individual cells may be combined or “pooled” prior to further sorting according to the same selection parameter or a different one, e.g. cells of a specific size can be first pooled before further sorting. Alternatively, the cells may be individually sorted, e.g. by single cell sorting. Such single cell sorting can be highly efficient providing for a fast production of the cell line.

Typically, cells are sorted into populations and subpopulations based on the presence or absence of a certain desired phenotype or physical appearance. Sorting allows capturing and collecting cells of interest for further cloning. Once collected, the isolated single cells can be expanded and cultivated, e.g. to finally select the cells which are capable of producing the POI at a high yield, and to prepare a master cell bank and optionally further prepare a working cell bank. Specifically, there is no need to prepare subclones or any re-cloning steps. The production cell line can be established immediately from a single clone and this cell line can be used to make-up the master cell bank. Cells from the master cell bank can be expanded to form a working cell bank, which is characterized for cell viability and proliferation prior to use in a POI manufacturing process.

The flow cytometry method simultaneously analyzing multiple physical characteristics of single cells is well-known in the art. Exemplary properties measured include cell size, relative granularity or internal complexity. The characteristics of each cell are e.g. based on its light scattering properties, which is analyzed to provide information about subpopulations within the sample.

Specifically, said sorting is by flow cytometry method using forward light scatter (FSC) and/or side light scatter (SSC).

In one embodiment, forward-scattered light and side-scattered light data are collected on the sorted cells. FSC is proportional to cell-surface area or size. As a measurement of mostly diffracted light, FSC provides a suitable method of detecting particles greater than a given size independent of their fluorescence. SSC is proportional to cell granularity or internal complexity, based on a measurement of mostly refracted and reflected light. Correlated measurements of FSC and SSC allows for differentiation of cell types in a heterogeneous cell population, without the necessity for staining or labeling the cell. The cells can be further sorted based on desired properties.

The cell sorting may be performed using devices which are typically used in fluorescence-activated cell sorting (FACS) or immunomagnetic cell sorting (MACS), preferably in a high-throughput and accurate way. In one embodiment, single cells are sorted directly into separate wells to produce individual clones.

Specific sorting techniques employ gating, which sets a numerical or graphical boundary to define the characteristics of cells to be included or excluded for further analysis. For example, a gate can be drawn around the population of interest. A gate or a region is a boundary drawn around a subpopulation to isolate events for analysis or sorting. Based on FSC or cell size, a gate can be set on the FSC versus SSC plot to allow analysis only of cells of a desired size and appearance. In one embodiment, recombinant host cells pre-selected by enrichment of cells under selective pressure are sorted by FSC/SSC gating, thereby obtaining a gated subpopulation that has the predetermined physical appearance or viability characteristics indicating genetic stability and an improved productivity.

Specifically, said sorting step is without using a label, such as a fluorescence label. Thus, the sorting step can avoid staining or labeling the repertoire of recombinant host cells.

Gating parameters may be based on cell intrinsic physical parameters only, and gates can be constructed based on a unique population, e.g., identified as larger and less granular than the majority of cells in the population. Specifically, the gating step comprises selecting sorted viable, recombinant host cells that possess a distinct physical profile (FSC/SSC population). The sorted cell culture wells of interest can then be harvested and further processed as described herein.

Once the single cells are sorted, typically, the sorted cells are separately grown, e.g. in wells or other separate containments, to obtain single clones during a time period of at least 1, 2, 3, 4, 5, 6, 7, 8, 9, or 10 days up to 8 weeks, 7, 6, 5, 4, 3 weeks, or less, e.g. up to 20, 19, 18, 17, 16, 15, 14, 13, 12, or 11 days. Such single clone cultivation may be performed under selective pressure or not. Afterwards, the clones may be analysed for cell culture performance, e.g. for POI productivity and/or the expression of the selection marker, before finally defining them as the production cell line. Generally, a supernatant containing the POI is collected, which can be analysed for the quantity and/or functionality of the POI.

According to a specific aspect, said repertoire of recombinant host cells comprises at least 10.000 different clones, or at least 10⁵, or at least 10⁶, or at least 10⁷, or at least 10⁸ different clones, or at least 10⁹ different clones, which differ in at least one genetic characteristic.

Specifically, said repertoire of recombinant host cells comprises a variety of copy numbers of said GOI, and wherein the variety of copy number ranges between 1 to 500. According to a specific embodiment, the cells of the repertoire comprise at least 5 or at least 10 or at least 15 or at least 20 copies of the GOI on the average. Specifically, a subpopulation of cells may be obtained which is characterized by a higher average copy number, e.g. where the average GOI copy number per cell is at least any of 5, 10, 15, 20, 25, 30, 35, 40, 45, or 50. A selected single cell is preferably characterized by a high GOI copy number, e.g. of at least or more than 5 or 10, or at least any of 15, 20, 25, 30, 35, 40, 45, 50, 55, 60, 65, 70, 75, 80, 85, 90, or 100.

Specifically, the single cell is selected from the repertoire of recombinant cells with a selection efficiency of at least 1 selected cell from a total of at least 10³, at least 10⁴, at least 10⁵, at least 10⁶, or at least 10⁷ recombinant cells, preferably wherein the selected cell is a high producer cell with a specific productivity of at least 1pcd, more preferably of at least 2, 5, 10, 15, 25, or 35 pcd, when specific productivity is already measured upon culture and production in static 96 well plates. Such high selection efficiency is a prerequisite for directly selecting transformants from a large population of cells, and in particular those of high productivity and genetic and epigenetic stability without the need of re-cloning or producing subclones which would provide a further repertoire of recombinant host cells that would need to be further screened for improved versions of the first selected clone. The selection efficiency can be highly improved without undue pre-selections or staged selections, in particular without serial dilutions and growing the clones under selective conditions.

According to a specific embodiment, said production cell line has a specific productivity producing the POI, of at least 0.1 pcd (pg/cell/day), preferably at least 1, 5, 10, 15, 20, 25, or 30 pcd under batch, fed-batch or continuous cultivation conditions, specifically during the production phase of a fed-batch culture. Specifically, the cultivation is performed in a bioreactor starting with a batch phase followed by a production phase allowing the production of the POI at a high yield.

Preferably, said production cell line is produced within less than 60 days, specifically, less than 50, or 40 days, or within a month, more specifically within 4 weeks, or even less than 4 weeks.

Specifically, said production cell line has a specific productivity producing the POI of at least 0.1 pcd, and said production cell line is produced within less than 60 days.

Specifically, the POI is a recombinant or heterologous protein, preferably any of a therapeutic protein, an immunogenic protein, a diagnostic protein or a biocatalyst. Specifically, the POI is selected from the group consisting of antibodies or fragments thereof, enzymes and peptides, protein antibiotics, toxins, toxin fusion proteins, carbohydrate—protein conjugates, structural proteins, regulatory proteins, vaccines and vaccine like proteins or particles, process enzymes, cell signaling and cell ligand binding proteins, growth factors, hormones and cytokines, protein antibiotics, structural proteins or a metabolite of a POI. Specifically, the POI is a “difficult to express” POI.

The invention further provides for a eukaryotic production cell line or a repertoire of recombinant host cells qualifying as eukaryotic production cell lines, obtainable by the method as described herein, wherein the production cell line is characterized by at least ten copies of the GOI incorporated into the chromosome of the cell, and a constitutive productivity of at least 0.1 pcd, preferably at least 1, 5, 10, 15, 25, or 30 pcd. Such repertoire is specifically not labeled by a fluorescence label.

The constitutive productivity indicates the fitness of the cell despite its transformation to become the recombinant host cell. Thus, the production cell line of constitutive productivity supports the robust manufacturing of the POI over a long production cycle. As a result, the productivity remains stable while growing and/or during the production phase in a fed-batch culture over a long period of time.

### FIGURES

FIG. 1 shows the strategy for an improved method of isolation of stable single clones in higher eukaryotic cells for production of recombinant proteins, which are of commercial interest. Of particular interest is this new strategy for production of recombinant proteins in industrially relevant mammalian or avian cells. Within 1 month after transfection and without any labeling of cells, stable production clones with high recombinant protein production can be generated, isolated, characterized and stored via cell banking.

FIG. 2A shows schematically the strategy to identify and sort the best production clones from a mixed population based solely on the cell intrinsic parameters of light scattering—Forward Scatter (FSC) and Side Scatter (SSC)—via flow cytometry.

FIG. 2B shows an example for setting the gates for selection of a total cell population in flow cytometry based on two control populations, one live cell population and one dead cell population of the respective mixed cell population to sort. In this example, the dead cells appear in gate “P1”, whereas the live cells appear in gate “P2” and can be positively selected for further cultivation.

FIG. 3 shows two examples, which prove the concept of the presented method. (a) The upper panel shows the generation and isolation of single clones based on FSC and SSC characteristics for an intracellular protein. This intracellular protein is green fluorescent protein (GFP), which allows monitoring the production and cellular content of the POI already during selection and enrichment of the respective clones. (b) The lower panel shows the generation and isolation of single clones based on FSC and SSC characteristics for a secreted protein. The secreted protein in this example is human FGF23. For each panel, the upper and the lower, on the left side the total population of cells with the SSC on the y-axis and the FSC on the x-axis, as well as the sort gate for live cells is displayed. In the middle, the sorted population is displayed, again with the SSC on the y-axis and FSC on the x-axis. On the right side, a histogram for the sorted cells is displayed, where the channel detecting the green fluorescence is on the x-axis, and the counts in the respective channels are on the y-axis. “Total population” indicates total cell population; “Sorted population” indicates live cells that were sorted into 96-well plate and “Histogram for GFP” indicates the intensity of GFP fluorescence along the x-axis and number of cell counts on the y-axis.

FIG. 4 shows a comparison of fluorescence intensity of single clones expressing GFP selected by different methods. The clones were selected either by high (1.0 mg/ml) or medium (0.5 mg/ml) antibiotics concentration and with the presented method of flow cytometry sorting, or they were classically generated by selection in pools and subsequently limiting dilution. All the clones were analysed by their GFP fluorescence intensity via flow cytometry, and the results of the fluorescence intensity for the population of single clones generated via the respective method is shown by three common statistical parameters “Mean”, Median”, and “Mode”.

FIG. 5 shows a comparison of specific productivity (pcd) distribution of single clones isolated by different methods for the example of FGF23 producing clones. The clones were selected either by high antibiotics concentration with the presented method of flow cytometry sorting, or they were classically generated by selection in pools and subsequently limiting dilution. In FIG. 5A the results for the clones are displayed in a box and whisker plot all three statistical parameters Mean, Median and Mode were used to plot the distribution of single clone pcd for each method tested. In FIG. 5B specific productivity is displayed using a scatter plot for visualizing the distribution of individual data point within the group. In both plots, the pcd values are plotted on the y-axis in a logarithmic scale from 0.01 to 100 pcds.

FIG. 6 shows a correlation between the volumetric yield (mg/l) and the specific productivity (pcd) for the single clones producing FGF23.

FIG. 7 shows the correlation between the gene copy number of the gene of interest and the gene copy number for the marker gene. In our example the GOI is FGF23, and the marker gene is neomycin resistance.

FIG. 8 shows a correlation between specific productivity and viability indicative for resistance to very high antibiotic concentrations of single clones. In FIG. 8A the resistance to G418 concentrations of 6 mg/ml was evaluated, in FIG. 8B the resistance to 10 mg/ml was evaluated.

FIG. 9 shows the fraction of transfected production cell line, which results in high production of the POI determined on the indicated day post transfection, and selection with 1 mg/ml G418 starting on day 1 post transfection. FIG. 9A sows the result when using the circular BAC, FIG. 9B shows the result when using linear BAC.

FIG. 10A: Vector map of a conventional plasmid-eGFP (used in Example 4 for the purpose of comparison) comprising the eGFP sequence driven by a the Caggs-promoter and an optimized Kozak-sequence just upstream of the eGFP start codon.

FIG. 10B: Vector map of a convention plasmid-FGF23 (used in Example 2) for construction of a BAC containing the FGF23 expression cassette in the Rosa26 locus (FGF23 (C-terminus) vector map).

FIG. 11: Sequences

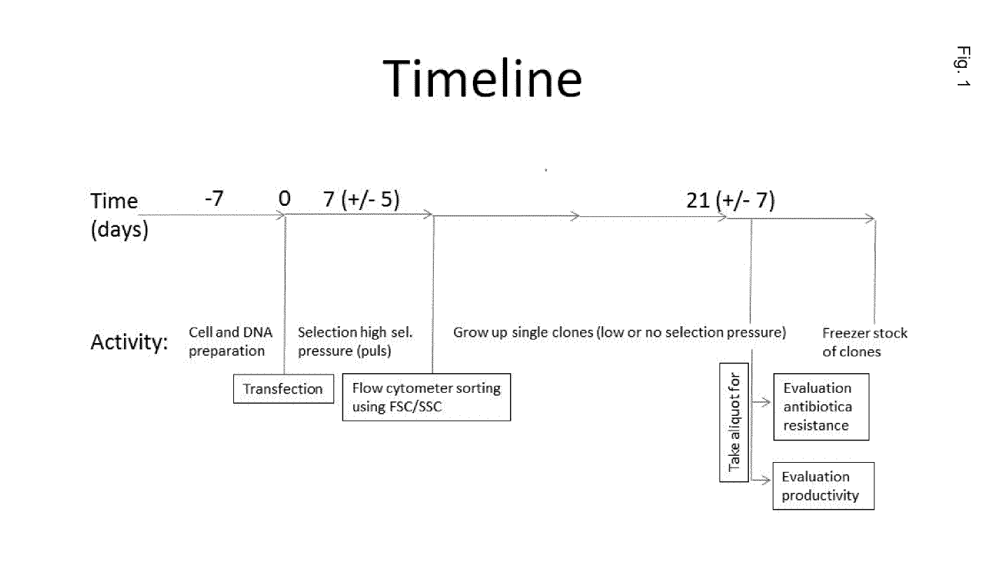

SEQ ID NO:5: Sequence of recombinant tagged human FGF23 (ctFGF23-His): c-terminal hFgF23 (180-251) protein sequence including leader sequence, short spacer and his tag; artificial sequence.

SEQ ID NO: 6: Sequence of plasmid-eGFP

SEQ ID NO: 15: Sequence of plasmid-FGF23

The sequence listing includes the following further sequences:

SEQ ID NO:1: Sequence of Rosa26 locus (corresponding to clone RPCI-24-85L15 (ID760448); GRCm38.p3 C57BL/6J: Chr. 6 (NC_000072.6): 112, 952, 746-113, 158, 583; source: NCBI), origin: mus musculus;

SEQ ID NO:2: Sequence of locus Rps21, (corresponding to clone RP23-88D12 (NCBI Clone Database ID:627270), origin: mus musculus.

SEQ ID NO:3: Sequence of locus Actb, (corresponding to clone RP23-5J14, (NCBI Clone Database ID:601738), origin: mus musculus.

SEQ ID NO:4: Sequence of locus Hprt, (corresponding to clone RP23-412J16 (NCBI Clone Database ID:732121;), origin: mus musculus.

### DETAILED DESCRIPTION OF THE INVENTION

Specific terms as used throughout the specification have the following meaning.

The term “artificial chromosome” as used herein refers to DNA molecules assembled in vitro from defined constituents, which enable stable maintenance of large DNA fragments with the properties of natural chromosomes. Artificial chromosomes usually contain elements derived from chromosomes that are responsible for replication and maintenance in the respective organism, and are capable of stably maintaining large genomic DNA fragments. In addition to replication origin sequences, the artificial chromosomes may have selection markers, usually antibiotic resistance markers, which allow the selection of cells carrying an artificial chromosome.

Artificial chromosomes are preferably derived from bacteria, like a bacterial artificial chromosome, also called “BAC”, e.g. having elements from the F-plasmid, or artificial chromosome with elements from the P1-plasmid, which are called “PAC”. Artificial chromosomes can also have elements from bacteriophages, like in the case of “cosmids”. Further artificial chromosomes are derived from yeast, like a yeast artificial chromosome, also called “YAC”, and from mammals, like a mammalian artificial chromosome, also called “MAC”, such as from humans and a human artificial chromosome, called “HAC”. Cosmids, BACs, and PACs have replication origins from bacteria, YACs have replication origins from yeast, MACs have replication origins of mammalian cells, and HACs have replication origins of human cells. Artificial chromosomes are usually in the range of 30-50 kb for cosmids, 50-350 kb for PACs and BACs, 100-3000 kb for YACs, and >1000 kb for MACs and HACs for their capacity to incorporate large DNA segments encompassing genes and their regulatory elements.

The term “cell line” as used herein refers to an established clone of a particular cell type that has acquired the ability to proliferate over a prolonged period of time. The term “production cell line” refers to a cell line as used for expressing an endogenous or recombinant gene or products of a metabolic pathway to produce polypeptides or cell metabolites mediated by such polypeptides. A production cell line is commonly understood to be a cell line ready-to-use for cultivation in a bioreactor to obtain the product of a production process, such as a POI. The production cell line can e.g. be provided as a master cell bank or working cell bank.

The term “cultivation”, also termed “fermentation”, with respect to a host cell line or production cell line is meant the maintenance of cells in an artificial, e.g., an in vitro environment, under conditions favoring growth, differentiation or continued viability, in an active or quiescent state, of the cells, specifically in a controlled bioreactor according to methods known in the industry. Specific cultivation media as used herein, in particular following the selecting step, are serum-free and contain no antibiotic or other drug which would confer selective conditions. The resulting master cell bank of the production cell line may thus be free of antibiotics. However, in some cases, selective conditions are maintained throughout the manufacturing process to obtain a master cell bank in a medium under selective pressure.

Cultivation of a production cell line and determination of its productivity can be performed in batch, fed-batch, or continuous processes, or semi-continuous process (e.g. chemostat). Whereas a batch process is a cultivation mode in which all the nutrients necessary for cultivation of the cells are contained in the initial culture medium, without additional supply of further nutrients during fermentation, in a fed-batch process, after a batch phase, a feeding phase takes place in which one or more nutrients are supplied to the culture by feeding. The purpose of nutrient feeding is to increase the amount of biomass in order to increase the amount of recombinant protein as well. Although in most cultivation processes the mode of feeding is critical and important, the present invention employing the promoter of the invention is not restricted with regard to a certain mode of cultivation.

The term “expanding” as used herein refers to an increase in number of viable cells derived from one single cell. Expanding may be accomplished by, e.g., “growing” a cell through one or more cell cycles, wherein at least a portion of the cells divide to produce additional cells.

As used herein, “coexpression” refers to expression of two or more nucleic acid sequences in the same cell. The level of expression of the two or more nucleic acid sequences may be the same or different. However, expression can be at a defined ratio, i.e. high expression of one nucleic acid sequence indicates high expression of the other nucleic acid sequence. Thus, expression of the two or more nucleic acids is correlated.

For example, the GOI and the selection marker gene can be expressed simultaneously, concurrently or sequentially in the same cell. High expression of the selection marker gene, for example assessed by resistance to a drug or toxin (e.g. an antibiotic), indicates that also the GOI is expressed at a high rate. In some embodiments, the GOI and selection marker genes are operably linked, and thereby coexpressed.

The term “euchromatin protein expression locus” is herein understood in the following way:

A locus (plural: loci) is the specific location or position of a gene or DNA sequence on a chromosome, in the field of genetics. A locus can be contained within a chromosomal segment that includes expression sequences which may be operable to express a gene. The locus as described herein is specifically a locus suitable for protein expression and characterized by a euchromatin structure.

Chromatin is a complex of macromolecules found in cells, consisting of DNA, protein and RNA. The primary functions of chromatin are 1) to package DNA into a smaller volume to fit in the cell, 2) to reinforce the DNA macromolecule to allow mitosis, 3) to prevent DNA damage, and 4) to control gene expression and DNA replication. The primary protein components of chromatin are histones that compact the DNA. The structure of chromatin depends on several factors. The overall structure depends on the stage of the cell cycle. During interphase, the chromatin is structurally loose to allow access to RNA and DNA polymerases that transcribe and replicate the DNA. The local structure of chromatin during interphase depends on the genes present on the DNA: DNA coding genes that are actively transcribed (“turned on”) are more loosely packaged in an open chromatin structure and are found associated with RNA polymerases (referred to as “euchromatin”), while DNA coding inactive genes (“turned off”) are found associated with structural proteins and are more tightly packaged (heterochromatin).

Specific loci in eukaryotic cells are particularly suitable for introducing a GOI or engineering expression constructs, which loci are characterized by the presence of euchromatin, and herein referred to as euchromatin protein expression loci. Exemplary loci which are characterized by euchromatin and described herein are any of Rosa26, Rps21, Actb, or Hprt and analogs of mammalian cells, such as human, mouse, hamster, dog, monkey, and in non-mammalian cells such as avian cells.

The chromatin structure and modifying elements are further described below:

A “chromatin element” means a nucleic acid sequence on a chromosome having the property to modify the chromatin structure when integrated into that chromosome. “Cis” refers to the placement of two or more elements (such as chromatin elements) on the same nucleic acid molecule (such as the same vector, plasmid or chromosome). “Trans” refers to the placement of two or more elements (such as chromatin elements) on two or more different nucleic acid molecules (such as on two vectors or two chromosomes). Chromatin modifying elements that are potentially capable of overcoming position effects, and hence are of interest for the development of stable cell lines, include antirepressors, boundary elements (BEs), matrix attachment regions (MARs), locus control regions (LCRs), and universal chromatin opening elements (UCOEs). Boundary elements (“BEs”), or insulator elements, define boundaries in chromatin in many cases and may play a role in defining a transcriptional domain in vivo. BEs lack intrinsic promoter/enhancer activity, but rather are thought to protect genes from the transcriptional influence of regulatory elements in the surrounding chromatin. Boundary elements have been shown to be able to protect stably transfected reporter genes against position effects in Drosophila, yeast and in mammalian cells. They have also been shown to increase the proportion of transgenic mice with inducible transgene expression. Locus control regions (“LCRs”) are cis-regulatory elements required for the initial chromatin activation of a locus and subsequent gene transcription in their native locations (Grosveld, F. 1999, “Activation by locus control regions” Curr Opin Genet Dev 9, 152-157). The activating function of LCRs also allows the expression of a coupled transgene in the appropriate tissue in transgenic mice, irrespective of the site of integration in the host genome. While LCRs generally confer tissue-specific levels of expression on linked genes, efficient expression in nearly all tissues in transgenic mice has been reported for a truncated human T-cell receptor LCR and a rat LAP LCR. The most extensively characterized LCR is that of the globin locus. “MARs”, according to a well-accepted model, may mediate the anchorage of specific DNA sequence to the nuclear matrix, generating chromatin loop domains that extend outwards from the heterochromatin cores.

The model of loop domain organization of eukaryotic chromosomes is well accepted. According to this model, chromatin is organized in loops that span 50-100 kb attached to the nuclear matrix, a proteinaceous network made up of RNPs and other non-histone proteins. The DNA regions attached to the nuclear matrix are termed SAR or MAR for respectively scaffold (during metaphase) or matrix (interphase) attachment regions. As such, these regions may define boundaries of independent chromatin domains, such that only the encompassing cis-regulatory elements control the expression of the genes within the domain. However, their ability to fully shield a chromosomal locus from nearby chromatin elements, and thus confer position-independent gene expression, has not been seen in stably transfected cells. On the other hand, MAR (or S/MAR) sequences have been shown to interact with enhancers to increase local chromatin accessibility. Specifically, MAR elements can enhance expression of heterologous genes in cell culture lines.

All the above elements contribute to confer epigenetic stability of an expression locus and perpetuate its expression activity state. The molecular basis of epigenetics is complex and involves modifications of the activation or inactivation of certain genes. Additionally, the chromatin proteins associated with DNA may be activated or silenced. When a cell divides, it must not only accurately duplicate its genome, but also restore its previous levels of gene expression. The information determining gene expression is often not directly encoded in the DNA and is hence termed ‘epigenetic’. The molecular basis of epigenetic memory arises at least from the collaboration of several mechanisms, including histone post-translational modifications, transcription factors, DNA methylation and noncoding RNAs. The term epigenetic stability as used herein refers to above mentioned mechanisms. The genetic and the epigenetic stability of the expression locus in the production cell line confer that the transcription levels for mRNA encoding the POI and for mRNA encoding the marker protein are not significantly altered (e.g. less than +/−50%, or 40%, or 30%, or 20%, or 10% variance) comparing their levels during the first 10 or 20 generations with their levels after 20 or 40 or 70 generations.

Chromosomal loci containing combinations of the above mentioned elements to keep the chromatin in an open or active state are thus providing an advantage for stable and constitutive expression of genes of interest. Such chromosomal loci can be adapted to form expression vectors. In order to amplify the DNA of such expression vectors, the chromosomal loci are generally combined with vector elements (herein referred to as “backbone”) to allow the rapid amplification of vector DNA in genetic organisms like bacteria or yeast. Such constructs are then called PAC, BAC, HAC, Cosmids or YAC.

A bacterial artificial chromosome (BAC) is typically a DNA construct, with a vector backbone based on a functional fertility plasmid (or F-plasmid), used for transforming and cloning in bacteria, usually E. coli. The bacterial artificial chromosome's usual insert size is 150-350 kbp, which can originate, for example, from mouse, hamster or human. A similar cloning vector called a PAC may be produced from the bacterial P1-plasmid.

Similarly, Yeast artificial chromosomes (YACs) are typically genetically engineered chromosomes derived from the DNA of the yeast. By inserting large fragments of DNA, from 100-1000 kb which can originate, for example, from mouse, hamster or human, the inserted sequences can be cloned and physically mapped. The primary components of the vector backbone of a YAC are the autonomously replicating sequence (ARS), centromere, and telomeres from S. cerevisiae. Additionally, selectable marker genes, such as antibiotic resistance and a visible marker, are utilized to select transformed yeast cells.

BAC-based vectors (and inter alia PAC and YAC) are specifically appropriate expression vectors for the purpose as described herein, because they can accommodate large eukaryotic genomic DNA inserts containing open chromatin regions or “hot spots”. This makes the BAC-based vectors insensitive to chromatin positional effects and confers them constitutive, copy number-dependent and predictable expression. Cell clones generated with BAC-based expression vectors typically contain several integrated copies of the BAC vector. This leads to a boost in the expression of the gene of interest straightforward after transfection and clone isolation, without subsequent rounds of transgene amplification. Consequently, BAC based vectors should carry chromatin regions or hot spots that allow high expression levels of the transgene. For example, the Rosa26 and housekeeping genes like the Hprt locus are considered to be hot spots.

The term “heterologous” refers to a nucleic acid e.g., a gene or regulatory element such as a promoter, refers to a nucleic acid occurring where it is not normally found or not naturally occurring, thereby engineering an artificial polynucleotide or nucleic acid. For example, a heterologous gene may be a native, wild-type, or mutant gene and linked to a nucleic acid sequence which is not normally found operably linked to the gene. Any gene that is an exogeneous gene, i.e. derived from a different organism or species, is a heterologous gene. Any exogenous locus, i.e. derived from a different organism or species, is a heterologous locus. A locus isolated from a cell and engineered to produce an expression construct is understood as artificial locus and exogenous to the source cell, even if it is re-introduced into the same cell or same type of cell. It is understood that the POI encoded by a heterologous GOI is considered as a heterologous POI.

The term “operably linked” as used herein refers to the association of nucleotide sequences on a single nucleic acid molecule, e.g. an expression cassette or construct, in a way such that the function of one or more nucleotide sequences is affected by at least one other nucleotide sequence present on said nucleic acid molecule. For example, a promoter is operably linked with a coding sequence of a recombinant gene, when it is capable of effecting the expression of that coding sequence. As a further example, a nucleic acid encoding a signal peptide is operably linked to a nucleic acid sequence encoding a POI, when it is capable of expressing a protein in the secreted form, such as a preform of a mature protein or the mature protein. Specifically such nucleic acids operably linked to each other may be immediately linked, i.e. without further elements or nucleic acid sequences in between the nucleic acid encoding the signal peptide and the nucleic acid sequence encoding a POI.

“Expression cassette” as used herein refers to nucleic acid sequences comprising a desired coding sequence and control sequences in operable linkage such that recombinant cells transformed or transfected with these sequences are capable of expressing the encoded protein. Expression cassettes frequently and preferably contain an assortment of restriction sites suitable for cleavage and insertion of desired coding sequence. An expression vector may contain one or more expression cassettes operable to express one or more genes.

An expression cassette as described herein specifically comprises a promoter operably linked to a desired coding sequence (or to a cloning site for a coding sequence) under the transcriptional control of said promoter.

In some embodiments, the expression cassette comprises a GOI, i.e. a nucleic acid sequence encoding a POI. Specifically, the GOI is a heterologous GOI. In some embodiments, the expression cassette comprises a coding sequence of a selection marker gene. In some embodiments, the expression cassette comprises both, a GOI and a selection marker gene, operably linking the GOI and the selection marker.

The term “expression construct” as used herein refers to a nucleic acid molecule comprising one or more expression cassettes. Expression constructs comprising more than one expression cassette may comprise expression cassettes with the same or different coding sequences and/or the same or different promoters. An expression construct may be a vector, plasmid or an artificial chromosome, in particular an artificial chromosome vector. The expression construct as used herein is incorporated into the host cell chromosome, and preferably not provided in a non-chromosomal location, e.g. as a plasmid. The stable incorporation into one or more chromosomes of the host cell renders the recombinant host cell genetically stable which facilitates the positive selection of high producer cells from the repertoire of recombinant host cells, thereby reducing the percentage of unstable transformants in the selection.

The procedures used to ligate the DNA sequences, e.g. coding for regulatory sequences, selection marker and/or the POI, respectively, and to insert them into suitable vectors containing the information necessary for integration or host replication, are well known to persons skilled in the art, e.g. described by J. Sambrook et al., “Molecular Cloning 2nd ed.”, Cold Spring Harbor Laboratory Press (1989). Specific techniques employ homologous recombination.

In some embodiments, the expression construct comprises one or more GOI expression cassettes. In some embodiments, the expression construct additionally comprises one or more selection marker gene expression cassettes. In some embodiments, the expression construct comprises the number of selection marker genes and GOI at a predefined ratio. For example, an expression construct may comprise one copy of a selection marker gene and any one of at least 1, 5, 10, 20, 30, 40, 50, 70, 100, 200, 300, 400 copies of a GOI.

As an example, an expression construct may comprise one copy of a selection marker gene and 10 copies of a GOI, thus providing the selection marker gene and the GOI at a predefined ratio of 1 to 10. In some embodiments, the expression construct comprises one or more expression cassettes with one copy of a GOI and one copy of a selection marker, thereby providing the selection marker gene and the GOI at a fixed or predefined rate of 1:1. For example, an expression construct may comprise any one of at least 1, 5, 10, 20, 30, 40, 50, 70, 100, 200, 300, 400 expression cassettes each comprising one copy of a selection marker gene and one copy of a GOI, whereby the predefined rate of selection marker gene to GOI is 1:1.

A “host cell” as used herein refers to a cell suitable for introduction of an expression construct and for expressing a protein of interest. Host cells are capable of growth and survival when placed in either monolayer culture or in suspension culture in a medium containing the appropriate nutrients and growth factors. Host cells can be eukaryotic cells, preferably mammalian cells (e.g. human, or rodent cells such as hamster, mouse or rat cells) or avian cells. In general, host cells can be any cell suitable for recombinant expression of a POI. Examples of preferred host cells are any one of the following:

Human production cell lines: HEK293, VERO, HeLa, Per.C6, VERO, HuNS1, U266, RPMI7932 (and derivative CHL),

Hamster cell lines: CHO, BHK, V79,

Derivatives thereof like preferably CHO-DG44, CHO-DUXB11, CHO-DUKX, CHODUKX B11, CHO-K1, CHO Pro-5, CHOK1SV, CHO/CERT2.20, CHO/CERT2.41, CHO-S, or B14AF28-G3 or preferably BHK21, BHK-TK−

Mouse cell lines: NIH3T3, NS0, SP2/0

Monkey cell lines: COS-7,

Dog cell line: MDCK

Avian cell line: EB66,

or the derivatives/progenies of any of the foregoing.

The term “intrinsic physical biomarker” or “intrinsic physical properties” is interchangeably used herein, refers to intrinsic physical cell properties which are directly measurable on or in the cell, without determining the function of the cell, e.g. determining an expression product or a reporter, and in particular without the use of staining techniques or a label, in particular without using a fluorescence label. A wide range of fluorophores are typically used as labels in flow cytometry, and specifically not used in the selection step as described herein. Fluorophores are typically attached to an antibody that recognizes a target on or in the cell; they may also be attached to a chemical entity with affinity for the cell membrane or another cellular structure. Such label would only determine the expression of the cellular target, but would not provide an indication of whether the cell has a normal physical appearance or function as a viable cell (independent of POI expression).

Intrinsic physical properties include, but are not limited to cell size, cell cytoplasmic granularity, polarizability, refractive index, cell membrane potential, cell shape, electrical impedance, density, deformability, magnetic susceptibility, and hydrodynamic properties.

In some embodiments of the methods described herein, the intrinsic physical property is cell cytoplasmic granularity, polarizability, refractive index and/or cell membrane potential.

“Cell size”, as used herein, refers to the volume of a cell and how much three-dimensional space it occupies. Cell size can be measured e.g. by flow cytometry using the forward scatter parameter. This parameter is a measurement of the amount of the laser beam that passes around the cell and gives a relative size for the cell. Using a known control or standard such as beads with a known size, the relative size of the cells based on the size of the control or standard can be measured. For example, the selected host cells as described herein can be within a range of 5-10 μm for small cells, or 10-20 μm for mid-sized cells, and 20-40 μm for large cells. In some embodiments, the selected host cell as described herein has a cell size that is at least 10%, 20%, 30%, 40% or 50% larger or smaller than a control value or a cell size within a range. The control can be the mean or median size of a live, dying or dead cell or cell population of the same cell sort or type as the selected host cell.

“Cell cytoplasmic granularity”, as used herein, refers to the spatial frequency of variation in the optical contrast/index of refraction within a cell. Cell cytoplasmic granularity may be visualized by microscopic analysis of cells following staining with a dye, such as Prussian blue. It can be measured e.g. by flow cytometry without using a dye by the side scatter parameter, which is a measurement of the amount of the laser beam that bounces off of particulates inside of the cell. For example, the selected host cells as described herein can be characterized by a cell cytoplasmic granularity which is 80%, 70%, 60%, 50% or less compared to a control. The control can be the mean or median granularity of a live, dying or dead cell or cell population of the same cell sort or type as the selected host cell. The ratios of the values for cell size (FSC) divided by cell granularity (SSC) are for live cells commonly 10% higher, more often 20%, 30%, 40%, 50%, or even 2×, 3×, 4×, 5× or 10× or more higher than the ratios of the FSC/SSC values for dying or dead cells.

“Polarizability”, as used herein, refers to the dynamical response of a cell to external fields. A dielectrophoretic field can be applied by a biodevice to align cells in a dimension-orientation sorter and/or to move size-sorted cells in a size-based sorter. This dielectrophoretic field can be defined as an electric field that varies spatially or is non-uniform where it is being applied to the particles (e.g. cells). Positive dielectrophoresis occurs when the particle (e.g. cell) is more polarizable than the medium (e.g., buffer solution) and results in the particle being drawn toward a region of higher field strength. A system operating in this way can be referred to as operating in a positive dielectrophoresis mode. Negative dielectrophoresis occurs when the particle is less polarizable than the medium and results in the particle being drawn toward a region of lesser field strength. A system operating in this way can be referred to as operating in a negative dielectrophoresis mode. Live (positive control) or dead (negative control) cells of the same sort or type as the cells to be selected are used to set up a system taking into account how the cells behave in the respective medium or buffer conditions. Whether cells are less or more polarizable in the experimental conditions depends on their state, i.e. alive or dead. Accordingly, the conditions will be set in such a way that the cells positively selected behave in terms of their polarizability like live cells or a subpopulation of live cells with advantageous characteristics. Using the above two control populations (live cells, or dying and dead cells), the settings of the system will be adjusted in a way, that first less than 5% of the dead cells is sorted and second more than 50% of the live cells are sorted. Depending on the separation efficiency and number of cells, the percentage for selecting the dying or dead cells can be reduced below 5%, and the percentage for selecting the living cells can be increased to more than 50%

The “refractive index” of a cell is herein understood as a dimensionless number that describes how light or any other radiation propagates through the cell. It is a measure of the light-bending ability of the cell. For example, for the selected host cells as described herein, a specific refractive index for either live cells (live cell index) or dead cells (dead cell index) can be characterized in the experimental buffer or medium conditions with control cells of the same sort, which are either live or dead. The changes in the refractive indices of cell surfaces enables efficient identification and separation of cells with significant differences in surface composition, such as live or dead cells. For example, the selected host cell as described herein can be characterized by a change of refractive index compared to a control, e.g. mean or median refractive index of a live or dead cell or cell population of the same sort or type, of at least 10%, 20%, 30%, 40% or at least 50%.

The term “cell membrane potential” is herein understood as the difference in electric potential between the interior and the exterior of a biological cell. Cell membrane potentials change in several ways with the physiologic state of the cell. Since the expenditure of metabolic energy is required to maintain potentials, the potential across the membrane of an injured or dying cell is decreased in magnitude. More specifically, changes in membrane potential occur, when cells are stressed due to the absence of marker gene expression and environmental conditions (such as cell culture media conditions containing antibiotics or lacking essential molecules), which require marker gene expression for cell survival and/or cell proliferation. Before, after, or during the incubation of the cell population with the culture medium containing for example an antibiotic cytotoxic in the absence of a selection marker, a representative characteristic of the cell membrane potential of a live cell population as well as of a dead cell population is detected as a reference characteristic. Since several of the methods used to detect changes in membrane potential are non-destructive, the processes may be used in combination with cell sorting to produce cell populations rich in cells with desired marker gene specificities while preserving cell viability. This detected characteristic is used to determine, whether individual cells in a mixed population are live cells, dying cells or dead cells. For example, the selected host cell described herein may behave in terms of the cell membrane potential (e.g. in terms of a representative characteristic of the cell membrane potential) like live cells or a subpopulation of live cells with advantageous characteristics. One method of measuring membrane potential involves a modification of the techniques employed in conventional electronic cell counters. In these devices, individual cells suspended in saline are passed through an orifice interposed between a pair of electrodes which maintain a current in the suspending solution. The passage of a cell through the orifice varies the conductivity of the solution, resulting in a detectable voltage pulse. The height of the pulse is indicative of cell volume. Since the membranes of cells with different membrane potential typically have different ionic conductivities, signals containing information indicative of variations in the ion conductivity of the membrane of individual cells passing through the orifice can be obtained using alternating current. These may be used to compare the membrane potentials of individual cells, e.g. with the aid of a pulse height analyzer. Cell membrane potential can be further measured, for example, by patch clamp techniques.

The term “cell shape” refers to the spatial form contour or appearance of a cell. For example, the selected host cells as described herein can be characterized by a cell shape which has generally a bigger size and/or a more uniform shape than a control cell or cell population, such as a dying or dead cell or cell population of the same sort or type. The cell shape can be determined by physical parameters like their light scattering behavior such as in flow cytometry, or by their dielectrophorectic force or by their acoustic radiation force.

The term “electrical impedance” as used herein refers to the properties of a physical object that oppose the flow of electrical current through it. The electrical impedance of biological matter, such as a cell gives information on their state (e.g. live or dead cell) or function. For example, the selected host cells as described herein can be characterized by an electrical impedance which is different to a control. The control can be the mean or median electrical impedance of a live, dying or dead cell or cell population of the same cell sort or type as the selected host cell. The selected host cell may have a difference in electrical impedance of at least 10%, 20%, 30%, 40% or 50% compared to a control. Splitting an initially uniform cell population into two aliquots, where in one aliquot cells are kept live and in the other aliquot cell death is induced, the effect of electrical impedance of live and dying or dead cells can be determined such as in a Coulter-type electrical impedance measurement. Cells, being poorly conductive particles, alter the effective cross-section of the conductive microchannel. As these cells are less conductive than the surrounding liquid medium, the electrical resistance across the channel increases, causing the electric current passing across the channel to briefly decrease, and the intensity of this decrease correlates with the cell being a live, dying or dead cell. By monitoring such pulses in electric current, the number of cells for a given volume of fluid can be detected and their status analysed. The size of the electric current change is related to the size of the particle, enabling a particle size distribution to be measured, which can be correlated to mobility, surface charge, and concentration of the particles.

The term “hydrodynamic properties” refer to the properties of a cell which arise from physical interactions of the cell with aqueous solvent, such as deformability, viscosity and sedimentation, which causes different movement in a liquid medium. Hydrodynamic properties can be used as parameter for continuous particle separation to identify and sort live cells in a population. Splitting an initially uniform cell population into two aliquots, where in one aliquot cells are kept live and in the other aliquot cell death is induced, the hydrodynamic properties of live, dying or dead cells can be determined and used as control values. Generally, the cell shape for live cells is bigger than for dying or dead cells, and by their combination of size and surface appearance they have a different movement in a symmetric or asymmetric liquid flow. This can be used for separation of live and dead cells using bifurcation of laminar flow around obstacles such as cells. For example, a host cell as described herein can be positively selected when displaying hydrodynamic properties of live cells or a subpopulation of live cells with advantageous characteristics. With methods such as “pinched flow fractionation” or “asymmetric pinched flow fractionation”, continuous separation of cells can be achieved (Takagi et al., Lab Chip 5:778 (2005)). Pinched flow fractionation (PFF) allows the continuous size separation of cells in a microchannel. This method is also advantageous in that it utilizes only the laminar flow profile inside a microchannel, and thus, complicated outer field control can be eliminated. To be more specific, liquids with and without cells are continuously introduced into a microchannel having a pinched segment, and cells are separated perpendicularly to the direction of flow according to their sizes by hydrodynamic force. In addition, separated particles can be collected independently by making multiple branch channels at the end of the pinched segment. In asymmetric pinched flow fractionation (AsPFF), microchannels are equipped with asymmetrically arranged multiple branch channels at the end of the pinched segment. With this microchannel, liquid flow in the pinched segment is asymmetrically distributed to each branch channel, and the difference in cell positions near one sidewall in the pinched segment can be effectively amplified. This enables precise separation of small cells by a relatively large-sized pinched segment.

According to the methods described herein a single cell is sorted according to physical intrinsic biomarkers of the host cell employing a predefined selection parameter. In some embodiments, the predefined selection parameter is a level and amount and in particular a threshold. The threshold can be a threshold percentile which is determined in relation to other (non-selected) cells of the repertoire or the whole repertoire. For example, the predefined selection parameter can refer to the percentile of cells above and/or below and/or around a target value (i.e. closest to the target value), where the target value is e.g. the median or mean value of a subpopulation of cells (e.g. control cells, in particular live cells as positive control, or dead cells as negative control), or of the whole population of cells, e.g. the whole repertoire of recombinant host cells.

In some embodiments, the predefined selection parameter refers to a minimum, maximum, mean, or median value. In some embodiments, the predefined selection parameter is a level, amount, range or threshold compared to a control. The control can be a calibration value or curve, minimum, maximum, mean or median values of a physical property (e.g. cell size, granularity, volume, refractive index, polarizability, density, elasticity, deformability, cell membrane potential, cell shape, hydrodynamic properties, light scattering, dielecrophoresis or magnetic susceptibility). The predefined selection parameter can be a relative value as compared to controls, such as live or dead cells or a respective cell population of the same sort or type as the selected host cell. The predefined selection parameter can also refer to a region, a range or gate for a population of cells with certain characteristics, such as a population of live cells or a population of cells within a threshold percentile (e.g. 10^{th} percentile of cells closest to a target value, e.g. a mean or median value of a physical property).

In some embodiments the predefined selection parameter is a percentile score of any one of 10^{th} percentile, 20^{th} percentile, 30^{th} percentile, 40^{th} percentile, 50^{th} percentile. 60^{th} percentile, 70^{th} percentile, 80^{th} percentile or 90^{th} percentile score. As an illustration, if a score is in the 90^{th} percentile, it is higher than 90% of the other scores. In some embodiments, the predefined selection parameter is percentage of cells defined as best hits, e.g. 5% or 10% of the cells which best match the predefined selection parameters, or 20% best hits, or 30%, as determined by a score system. A score can be based on one or more cell intrinsic physical properties. In some embodiments, a score is based on cell size and cell granularity (e.g. a minimum, maximum or average cell size/cell granularity).

Several methods for measuring cell intrinsic physical properties, i.e., physical appearance are known in the art including, but not limited to methods based on microscale filters, hydrodynamic filtration, deterministic lateral displacement, field-flow fractionation, microstructures, inertial microfluidics, gravity, biomimetic microfluidics, magnetophoresis, aqueous two-phase systems, acoustophoresis, dielectrophoresis, optics, droplet-based microfluidics, raman-activated techniques, flow cytometry methods.

In the methods described herein, a single cell can be selected by sorting using an optical flow cytometry method or microfluidic systems—such as droplet based microfluidics or Raman-activated cell sorting or applying acoustic radiation force—according to physical differences in the properties of cells including size, shape, volume, density, elasticity, hydrodynamic property, polarizability, light scattering, dielectrophoresis, and magnetic susceptibility.

Cells can be separated in the dielectric separation method for example in three-dimensional (3D) nonuniform electric fields generated by employing a periodic array of discrete but locally asymmetric triangular bottom microelectrodes and a continuous top electrode (Ling et al. Microelectrode Array; Anal. Chem. 84 (15), pp 6463-6470 (2012)). Traversing through the microelectrodes, heterogeneous cells are electrically polarized to experience different strengths of positive dielectrophoretic forces, in response to the 3D nonuniform electric fields. The cells that experience stronger positive dielectrophoresis are streamed further in the perpendicular direction to the fluid flow, leaving the cells that experience weak positive dielectrophoresis, which continue to traverse the microelectrode array essentially along the laminar flow streamlines.

When cells suspended in fluid are exposed to ultrasound and a pressure amplitude, they experience an acoustic radiation force. Separation of particles utilizing this force can be achieved by generating a standing wave over the cross section of a microfluidic channel (Gossett et al. Anal Bioanal Chem: 397:3249-3267 (2010)). In this configuration, while a fluid carries cells through the channel, a radiation force pushes cells towards either the pressure nodes or the pressure antinodes of the standing wave. The strength of the acoustic radiation force depends on three different properties: the volume of the cell, the relative density of the cell and the fluid, and the relative compressibility of the cell and the fluid. The acoustic force can have the opposite sign for cells with different densities. These cells will be attracted to different parts of the channel: pressure nodes (high density cells) or antinodes (low density cells). Typically the focused cells are collected through a centered outlet while other particles exit from other outlets.

Raman analysis is a non-invasive method to acquire the chemical fingerprint of the whole single-cell without the need of labeling, identifying rapidly cell properties such as single-cell genotypes, physiological states and metabolite changes. The information of the targeted cells/particles can be identified and analyzed by the Raman spectra, the Raman spectroscopy data can be analyzed automatically and the switching device for sorting cells can be controlled by computer. The specific cells can be controlled using technical means including optical, magnetic or electric field, and the cells can be sorted into the different microfluidic channels by the microfluidic device. Therefore, it is well suited to isolate individual living cells from a population of dying or dead cells.

Droplet-based microfluidics as a subcategory of microfluidics in contrast with continuous microfluidics has the distinction of manipulating discrete volumes of fluids in immiscible phases with low Reynolds number and laminar flow regimes. Microdroplets offer the feasibility of handling miniature volumes of fluids conveniently, provide better mixing and are suitable for high throughput experiments. One of the key advantages of droplet-based microfluidics is the ability to use droplets as incubators for single cells. Devices capable of generating thousands of droplets per second opens new ways characterize cell population based on a specific marker or intrinsic cell property measured at a specific time point, or also based on cells kinetic behavior such as protein secretion or enzyme activity or proliferation.

When using flow cytometry cells may be sorted and selected based on FSC and/or SSC plots employing gates. A skilled person can employ general FACS techniques, e.g. using a population of living cells and defining a gate around them. Then one can use a population of dying or dead cells and check the gate setting, that those cells are not (or just accidentally) within the “living” gate. Thus, in the sample to be analysed and sorted, living cells would fall into the predefined gate, whereas the dead or dying cells would be outside this gate and discarded. In some embodiments, a host cell as described herein is selected if it falls within the gate for live cells or within a live cell population with advantageous characteristics. Such characteristics could be a particular subgate within the live cell gate, which defines a more narrow range for cell size and/or cell granularity (FSC/SSC). By evaluating the protein production characteristics of cells sorted by different narrow subgates within the live cell gate has the potential to identify a particular subgate, where the most productive cells can be found in higher frequency.

For example, the selection of cells (population of interest) to be sorted can be in the same gate in a FSC/SSC plot as those of a healthy proliferating control population. The starving or dying cells can be shifted to a lower FSC and higher SSC area and thus are mainly found outside of the sort gate. For setting the gate for a repertoire of host cells, two control or standard populations (one healthy, one dying) of the host cell line of the same type (but without being transfected, or just mock transfectants) are required. In a typical setting for a FACS Aria III Flow Cytometer from Becton Dickinson (as used in the present example below), the Voltage setting would be 140V for FSC-A and 250V for SSC-A. In a FSC/SSC plot (FSC-A on the x-axis, SSC-A on the y-axis), the asymmetric live gate is between 60 and 250 units in the FSC, and between 10 and 150 units in the SSC, starting narrow on the left bottom side and getting broader to the right and upper side. In general, the live cells to be sorted show about 110% or higher values for FSC-A, and only 90% or lower values (excluding the debris) for SSC-A.

The term “isolating” as used herein is defined as the process of releasing and obtaining a single cell from a mixture or collection of cells. An isolated cell is then separated from its original environment such as a cell culture, a repertoire of host cells transfected with an expression construct, a fraction of said repertoire of host cells (e.g. a fraction of pre-selected cells resistant to a drug), or a pool of cells selected based on their cell intrinsic properties, in particular their physical appearance. An isolation procedure described herein may involve the isolation of a single cell which was selected by sorting according to physical appearance.

The term “gene of interest” or GOI as used herein refers to a nucleic acid or polynucleotide or nucleotide sequence encoding the POI. The gene specifically may be a wild-type gene including introns or an open reading frame, or a codon-optimized or mutant gene.

The term “protein of interest” or POI as used herein refers to a polypeptide or a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, upon integration by recombinant techniques of one or more copies of the GOI into the genome of the recombinant cell, or by recombinant modification of one or more regulatory sequences controlling the expression of the gene encoding the POI, e.g. of the promoter sequence. In some cases the term POI as used herein also refers to any metabolite product by the recombinant cell as mediated by the recombinantly expressed protein.

The POI can be any eukaryotic, prokaryotic or synthetic polypeptide, and is particularly heterologous to the host cell. It can be a secreted protein or an intracellular protein, preferably for therapeutic, prophylactic, diagnostic, analytic or industrial use.

Specifically, the POI as described herein is a eukaryotic protein, preferably a mammalian protein, specifically a mammalian or human protein heterologous to the host cell.

Specifically, the POI is a single or multi-chain protein, including e.g. covalently (e.g. via binding bridges, or disulfide linked) or non-covalently linked homo- or heteromers of polypeptide chains.

According to one aspect of the invention, the POI is a recombinant or heterologous protein, preferably selected from therapeutic proteins, including antibodies or fragments thereof, enzymes and peptides, protein antibiotics, toxin fusion proteins, carbohydrate—protein conjugates, structural proteins, regulatory proteins, vaccines and vaccine like proteins or particles, process enzymes, growth factors, hormones and cytokines, or a metabolite of a POI.

Examples of preferably produced proteins are immunoglobulins, immunoglobulin fragments, aprotinin, tissue factor pathway inhibitor or other protease inhibitors, and insulin or insulin precursors, insulin analogues, growth hormones, interleukins, tissue plasminogen activator, transforming growth factor a or b, glucagon, glucagon-like peptide 1 (GLP-1), glucagon-like peptide 2 (GLP-2), GRPP, Factor VII, Factor VIII, Factor XIII, platelet-derived growth factor1, serum albumin, enzymes, such as lipases or proteases, or a functional homolog, functional equivalent variant, derivative and biologically active fragment with a similar function as the native protein.

The POI may be a native (wild-type) protein or structurally similar to the native protein and may be derived from the native protein by addition of one or more amino acids to either or both the C- and N-terminal end or the side-chain of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Such modifications are well known for several of the proteins mentioned above.

A POI can also be selected from substrates, enzymes, inhibitors or cofactors that provide for biochemical reactions in the host cell, with the aim to obtain the product of said biochemical reaction or a cascade of several reactions, e.g. to obtain a metabolite of the host cell. Exemplary products can be vitamins, such as riboflavin, organic acids, and alcohols, which can be obtained with increased yields following the expression of a recombinant protein or a POI according to the invention.

A POI produced according to the invention may be a multimeric protein, preferably a dimer or tetramer.

A specific POI is an antigen binding molecule such as an antibody, or a fragment thereof. The term “antibody” as used herein shall always include antigen-binding fragments thereof or domains of such antibodies. Among specific POIs are antibodies such as monoclonal antibodies (mAbs), immunoglobulin (Ig) or immunoglobulin class G (IgG), heavy-chain antibodies (HcAb's), or fragments thereof such as fragment-antigen binding (Fab), Fd, single-chain variable fragment (scFv), or engineered variants thereof such as for example Fv dimers (diabodies), Fv trimers (triabodies), Fv tetramers, or minibodies and single-domain antibodies like VH or VHH or V-NAR.

According to one embodiment, the POI is a “difficult to express” protein, herein also referred to as “difficult POI”, which is meant to be difficult to be expressed in heterologous expression systems. Such proteins typically require the expression of more than one polypeptide chains and/or specific folding by the host cell and/or post-translational modifications, e.g. glycosylation or phosphorylation, to render the protein functional. In a host cell factors such as codon usage, translation rate, and redox potential can have a significant impact on its capability to express such difficult POI. Exemplary difficult POI are selected from the group consisting of antibodies, viral envelop proteins, cytokines, cell surface receptors or parts thereof.

The term “recombinant” as used herein shall mean “being prepared by or the result of genetic engineering”. Thus, “recombinant nucleic acid” refers to nucleic acid formed in vitro by the manipulation of nucleic acid into a form not normally found in nature. A “recombinant protein” is produced by expressing a respective recombinant nucleic acid. A “recombinant cell” specifically has been genetically engineered to contain at least one recombinant nucleic acid sequence. A “recombinant host cell” is a host cell comprising a heterologous nucleic sequence, and is typically transformed with an expression construct to become recombinant.

As used herein, the term “repertoire” refers to a mixture or collection of diverse host cells which result from transfecting a host cell line with the same expression construct, i.e. the same GOI and/or selection marker, and differ in at least one genetic characteristic. The members of the repertoire of host cells are not all identical and within the repertoire can be distinguished e.g. by any one of or at least one of the (i) copy number of the GOI and/or selection marker, (ii) the site of integration of the GOI and/or selection marker into the chromosome, (iii) the genetic stability, and (iv) the epigenetic stability.

For example, a repertoire of host cells may comprise host cells with varying copy numbers of an expression cassette or construct, e.g. varying within the range of 1-500 copy numbers, e.g. on the average 1, 5, 10, 20, 30, 40, 50, 60, 70, 80, 90, or 100; or may include a fraction (or collection) of cells with at least any one of 5, 10, 20, 30, 40, 50, 60, 70, 80, 90, 100, 200, 300, 400, or 500 copy numbers.

The repertoire may include e.g. host cells with one or more expression cassettes or expression constructs incorporated at a number of different sites ranging between 1-100, e.g. 1-5 or 1-20 different loci, e.g. on the average 1, 5, 10, 20, 30, 40, or 50 different loci; or may include a fraction (or collection) of cells with at least any one of 1, 2, 3, 4, 5, 6, 7, 8, 9 or 10 different chromosomal sites in the host cell.

The repertoire may include e.g. host cells with varying genetic stability. “Genetic stability” as used herein refers to the maintenance of the recombinant nucleic acid, and in particular the number of expression constructs, incorporated in the host cell over a predetermined period of time in the cell culture. A repertoire of host cells with a variety of genetic stability thus comprises host cells which maintain their recombinant nucleic acid within a range of 5-70 generations, thus during a time period reflecting the respective multiplicity of the generation time, e.g. on the average 5, 10, 20, 30, 40, 50, or 70 generations; or may include a fraction (or collection) of cells with at least any one of 10, 20, 30, 40, 50 or 70 generations.

The term “epigenetic stability” as used herein shall refer to the epigenetic stability of the expression locus, which determines that the transcription levels for mRNA encoding the POI and for mRNA encoding the marker protein are not significantly altered (e.g. less than +/−50%, or 40%, or 30%, or 20%, or 10% variance) comparing their levels during the first 10 or 20 generations with their levels after 20 or 40 or 70 generations. This can be determined by measuring the mRNA levels for the GOI transcripts by quantitative RT-PCR and normalizing it to the mRNA levels for a housekeeping gene like Rps21.

A repertoire of host cells is obtainable either by random incorporation of a recombinant nucleic acid or site-directed incorporation, e.g. homologous recombination or targeted gene integration into site-specific loci using CRISPR/Cas9 genome editing system. The repertoire of host cells as described herein specifically refers to the whole cell population which was successfully transfected with the expression construct and is characterized by specific beneficial features of the cell which are suitable for the use of the cell in the development of a production cell line.

When a repertoire of host cells is obtained by incorporating an expression construct comprising one or more GOI expression cassettes wherein a selection marker gene is operably linked to the GOI, the expression construct can be incorporated at a variety of chromosomal loci and/or a variety of copy numbers. In this case, expression of the GOI and the selection marker can be at a predefined rate. Thus, the expression level of the selection marker can be indicative of the GOI expression level and the productivity of the POI production host cell.

When a repertoire of host cells is obtained by incorporating one expression construct comprising a defined number of selection marker expression cassette and independently one or more GOI expression cassettes, the selection marker expression can be indicative of the successful transfer of the construct into the host cell chromosome. Depending on whether the ratio of the expression of the selection marker and the POI is predetermined or varying, the selection marker can as well be indicative of the level of GOI expression or not.

When a repertoire of host cells is obtained by incorporating an expression construct comprising a GOI expression cassette and separately incorporating an expression construct comprising a selection marker expression cassette, the repertoire of host cells may include host cells with either one of the two expression constructs or both incorporated at a variety of copy numbers at a variety of chromosomal loci. In this exemplary case, expression of the GOI and the selection marker gene is not correlated.

A “selectable marker gene” or “selection marker gene” refers to a gene conferring a phenotype which allows the organism expressing the gene to survive under selective conditions. The gene specifically encodes the selection marker, and may be a wild-type gene including introns, or a codon-optimized or mutant gene.

Cells can proliferate under selective conditions if they are capable of overcoming a shortage of specific factors or if they can resist the otherwise detrimental effects of a drug. Cells which proliferate under selective conditions (herein also referred to as “selection resistant cells” or simply “resistant cells”) can supplement a missing metabolic function or have property of growing despite the presence of a drug, e.g. an antibiotic. For example, the selection marker gene can include one or more genes conferring the ability to grow in the presence of a drug, that otherwise would kill the cell. According to a further example, the selection resistant cell has the ability to grow in the absence of a particular nutrient, e.g. the ability to grow on a medium devoid of a necessary nutrient that cannot be produced by a deficient and untransformed cell, or the ability to grow on medium, e.g., an energy source, that cannot be used/metabolized by a deficient and untransformed cell.

Selection marker genes thus include one or more genes conferring resistance to a drug, e.g. an antibiotic (hereinafter referred to as “antibiotic resistance marker gene”), and marker genes conferring a metabolic function (hereinafter referred to as “metabolic function marker gene”).

In case of antibiotic resistance marker genes, only cells which have been transformed or transfected with this gene are able to grow in the presence of the corresponding antibiotic and are thus selected. For example, in order to select for the presence of an expressed antibiotic resistance gene such as neomycin phosphotransferase, the antibiotic geneticin (G418) is preferably used as the medium additive.

Exemplary antibiotic resistance marker genes that can be used as a genetic marker for eukaryotic cells include, but are not limited to (i) any aminoglycoside resistance marker genes such as genes conferring resistance to neomycin (G418), geneticin, kanamycin, streptomycin, gentamicin, tobramycin, neomycin B (framycetin), sisomicin, amikacin, and isepamicin, and hygromycin B; (ii) genes conferring resistance to puromycin; (iii) genes conferring resistance to bleomycines, preferably bleomycin, phleomycin or zeocin; (iv) blasticidin; or; (v) mycophenolic acid.

According to the methods described herein, selective conditions are obtained upon addition of the antibiotics to the cell culture medium following transfection with the expression construct to introduce the corresponding selection marker gene product into the host cell. Such method of selection for antibiotic resistance indicative of successful gene transfer into the recombinant host cell is well-known in the art and is well-described in the standard lab manuals. The repertoire of host cells as described herein is then grown (e.g., in the presence of the antibiotic) for at least any one of 1 day, 2 days, 3 days, 4 days, 5 days, 6 days, 7 days, 10 days or up to 12 days, under selective conditions expressing the selection marker gene and the GOI. Alternatively, the repertoire of host cells as described herein is kept under cultivating or maintenance conditions (e.g., under selective conditions expressing the antibiotic selection marker gene and the GOI in the presence of the antibiotic) for at most any one of 7 days, 6 days, 5 days, 3 days, 2 days, or 1 day.

According to a specific embodiment, the repertoire of host cells is first prepared and then kept in the pool under antibiotic selection pressure, e.g. by adding the antibiotic to the pool medium, such that more than 70%, 80% or 90% of the cells in the pool are killed. The antibiotic selection pressure is then removed, e.g. after 1, 2, 3, 4, 5, or 6 days of antibiotic selection pressure by exchanging or diluting the pool medium. The single cell sorting is then performed under low or no antibiotic selection pressure.

In the following, the various antibiotics and selective conditions for cells bearing the antibiotic resistant genes are described.

Aminoglycoside antibiotics comprise at least one amino-pyranose or amino-furanose moiety linked via a glycosidic bond to the other half of the molecule. Their antibiotic effect is based on inhibition of protein synthesis. Aminoglycoside resistance genes are commonly employed in the molecular biology of eukaryotic cells and are described in many standard textbooks and lab manuals. The aminoglycoside resistance gene product is reported to be a functional gene product in view of its aminoglycoside-degrading activity. Aminoglycoside resistance marker genes thus further include functional variants of known aminoglycoside resistance genes, i.e. gene products of variant resistance marker genes with aminoglycoside-degrading activity.

The aminoglycoside can be employed in a concentration of at least 0.01 mg/ml or at least 0.1 mg/ml, preferably in a concentration of at least 1 mg/ml, most preferably in a concentration of at least 4 mg/ml. In a further particularly preferred embodiment, aminoglycoside is employed in a concentration of 10 μg/ml to 400 μg/ml, preferably at a concentration of 1 to 4 mg/ml. Hygromycin B is an aminoglycoside antibiotic, which is employed in a concentration of at least 10 μg/ml, preferably 10 μg/ml to 400 μg/ml.

Puromycin is an antibiotic, which is employed in a concentration of at least 0.5 μg/ml, preferably 0.5 μg/ml to 10 μg/ml. Bleomycin, zeocin and phleomycin are glycopeptide antibiotics, which are employed as follows: Bleomycin is employed in a concentration of at least 50 μg/ml, preferably 50 μg/ml to 200 μg/ml. Zeocin is employed in a concentration of at least 0.1 mg/ml, preferably 0.1 to 0.4 mg/ml. Phleomycin is employed in a concentration of at least 0.1 μg/ml, preferably 0.1 μg/ml to 50 μg/ml. Blasticidin is a nucleoside antibiotic employed in a concentration of at least 2 μg/ml, preferably 2 μg/ml-10 μg/ml. Mycophenolic acid is employed in a concentration of at least 25 μg/ml.

In some embodiments, the selection marker gene is a neomycin phosphotransferase gene (e.g., neo from Tn5 encodes an aminoglycosidase 3′-phosphotransferase, ATP 3′II), KanMX (a hybrid gene consisting of a bacterial aminoglycoside phosphotransferase under control of the TEF promoter from Ashbya gossipii), hygromycin B phosphotransferase gene, puromycin-N-acetyltransferase (pac) gene, histidinol dehydrogenase, bleomycin resistance gene, bls (an acetyltransferase) from Streptoverticillum sp, bsr (a blasticidin-S deaminase) from Bacillus cereus, BSD (another deaminase) from Aspergillus terreus and Streptoalloteichus hindustanus (SH) ble gene, or functional variants of the above listed genes.

Preferably, the resistance gene product according to the present invention is a Neomycin-Phosphotransferase (the resistance gene commonly known as Neo′). Selection with G418 (Geneticine, as defined under Chemical abstracts Registry Number 49863-47-0) or Neomycin can be used to select for cells expressing the neomycin resistance gene product.

Exemplary metabolic function marker genes include, but are not limited to adenosine deaminase (ADA), dihydrofolate reductase (DHFR), glutamine synthetase (GS), histidinol D, thymidine kinase (TK), xanthine-guanine phosphoribosyltransferase (XGPRT), and cytosine deaminase (CDA).

Metabolic function marker genes may be dominant or recessive marker genes. Recessive marker genes require a particular host which is deficient in the activity under selection. Dominant marker genes function independent of the host.

Several recessive metabolic function marker genes are involved in the salvage pathway pyrimidine or purine biosynthesis. When the de novo pyrimidine or purine biosynthesis is inhibited, the cell can utilize salvage pathways using respective enzymes (e.g. thymdine kinase, xanthin-guanine-phosphoribosyltransferase, adenine phosphoribosyltransferase or adenosine kinase) necessary for conversion of nucleoside precursors to the respective nucleotides. These salvage pathways are not required for cell growth when de novo purine and pyrimidine biosynthesis are functional. Cells deficient of a salvage pathway enzyme are viable under normal growth conditions, but addition of drugs that inhibit de novo biosynthesis of purines or pyrimidines results in death of deficient cells because the salvage pathway becomes essential.

For example, thymidine kinase negative cells can be transfected with the thymidine kinase selection marker gene. When growing these cells under selective conditions, e.g. in a medium containing methotrexate or aminopterin, which inhibit the enzyme dihydrofolate reductase thus blocking the de novo synthesis of thymidine monophosphate, cells which have been successfully transfected, i.e. contain the thymidine kinase marker gene, survive and can be selected. A commonly used medium providing selective conditions for thymidine kinase is HAT medium, which contains hypoxanthine aminopterin and thymidine. Such selective medium for thymidine kinase is usually complete medium supplemented with 100 μM hypoxanthine, 0.4 μM aminopterin, 16 μM thymidine and 3 μM glycine.

Cells producing E. coli XGPRT can synthesize guanosine monophosphate (GMP) from xanthine via xanthine monophosphate (XMP). After transfection with XGPRT selection marker, surviving cells producing XGPRT can be selectively grown with xanthine as the sole precursor for guanine nucleotide formation in a medium containing inhibitors (aminopterin and mycophenolic acid) that block de novo purine nucleotide synthesis. Such selective medium generally contains dialyzed fetal bovine serum, 250 μg/ml xanthine, 15 μg/ml hypoxanthine, 10 μg/ml thymidine, 2 μg/ml aminopterin, 25 μg/ml mycophenolic acid and 150 μg/ml L-glutamine.

Cytosine deaminase is a non-mammalian enzyme, which catalyzes the deamination of cytosine and 5-fluorocytosine to form uracil and 5-fluorouracil, respectively. Inhibition of the pyrimidine de novo synthesis pathway creates a condition in which cells are dependent on the conversion of pyrimidine supplements to uracil by cytosine deaminase. Thus, only cells expressing the cytosine deaminase gene can be rescued in a respective selection medium, usually containing 1 mM N-(phosphonacetyl)-L-aspartate, 1 mg/ml inosine, and 1 mM cytosine.

The dihydrofolate reductase (DHFR) is required for the biosynthesis of glycine from serine, thymidine monophosphate from deoxyuridine-monophosphate and for the biosynthesis of purine. DHFR deficient cells require the addition of thymidine, glycine and hypoxanthine and do not grow in the absence of added nucleosides unless they acquire a functional DHFR gene. Methotrexate (MTX), a folate analogue, binds to and inhibits the dihydrofolate reductase and thus causes the cell death of the exposed cells. Cells are selected for growth with increasing or high MTX concentrations (e.g. 0.01 to 300 μM MTX), requiring the surviving cells to contain increased levels of DHFR.

Glutamine synthetase (GS) is the enzyme responsible for the biosynthesis of glutamine from glutamate and ammonia. This enzymatic reaction provides the only pathway for glutamine formation in a mammalian cell. In the absence of glutamine in the growth medium, the GS enzyme is essential for the survival of mammalian cells in culture. Some mammalian cell lines, such as mouse myeloma lines, do not express sufficient GS to survive without added glutamine. With these cell lines, a transfected GS gene can function as a selectable marker by permitting growth in a glutamine-free medium. Other cell lines, such as Chinese hamster ovary (CHO) cell lines, express sufficient GS to survive without exogenous glutamine. In these cases, a GS inhibitor, e.g., methionine sulphoximine (MSX used at a concentration between 10 μM to 70 μM), can be used to inhibit endogenous GS activity such that only transfectants with additional GS activity can survive. GS can thus be used as selection marker using culture medium without glutamine either (i) in GS deficient host cells, natively deficient or deletion of gene or (ii) or cells with GS function and a GS inhibitor.

Adenosine deaminase (ADA) is present in virtually all mammalian cells and is not an essential enzyme for cell growth. ADA catalyzes the irreversible conversion of cytotoxic adenosine nucelosides to their respective nontoxic inosine analoges. Cells propagated in the presence of cytotoxic concentrations of adenosine or cytotoxic adenosin analogues such as 9-D-xylofuranosyl adenine (XylA) require ADA to detoxify the cytotoxic agent. 2′-deoxycoformycin (dCF), a tight binding transition state analogue inhibitor of ADA can be used to select for amplification of the ADA gene, using concentrations of 0.01 to 0.3 μM dCF. As a selective media for ADA a medium containing 10 μg/ml thymidine, 15 μg/ml hypoxanthine, 4 μM 9-β-D-xylofuranosyl adenine can be used.

The Salmonella typhimurium gene hisD encodes the protein histidinol dehydrogenase, which catalyzes the conversion of histidinol to the amino acid histidine. Histidinol is toxic to mammalian cells, while histidine is an essential mammalian amino acid. Consequently, growth selection in cultures with media containing histidinol in place of histidine occurs by both histidine starvation and histidinol poisoning. Typical selection conditions are provided by a medium containing 1 mM N-(phosphonacetyl)-L-aspartate, 1 mg/ml inosine, and 1 mM cytosine.

Selective conditions may also trigger amplification of the selectable marker gene if the gene used is an amplifiable selectable marker gene. Methotrexate, for example, is a selecting medium which is suitable for amplifying the DHFR gene. 2′-deoxycoformycin (dCF) can be used for amplifying the ADA gene.

The term “high selective pressure” means selection under high stringency, e.g. very high antibiotic concentration in the culture medium (e.g. at least 1 mg G418 per ml of ml culture medium). High stringency means selection pressure that will remove, kill, make distinguishable or selectable more than 90%, preferably more than 99%, even more preferably more than 99.9%, most preferable 99.99% of cells that have been subjected to transfection so that the remaining small fraction represents the successfully transfected clones with the highest expression level. Most preferably the selection pressure will be employed on the transfected cells within less than 3 days to obtain a repertoire of surviving or robust cells. In some embodiments, the repertoire of cells is selected for single cells immediately after subjecting the transfectants to a high selective pressure, and the single cell sorting is followed by cultivation of sorted cells under low or no selective pressure, i.e. wherein at least 50% of the sorted cells, preferably at least 40%, or at least 30%, or at least 20%, or at least 10%, or at least 1% survive the selective pressure.

“Transformation” and “transfection” are used interchangeably to refer to the process of introducing DNA into a cell.

According to the methods described herein, an expression construct is incorporated into the chromosome of the host cell, thereby obtaining a repertoire of host cells. The expression construct can thereby either be randomly incorporated or integrated at a specific site.

The term “randomly incorporated” refers to integration of a nucleic acid, at unspecified sites of a chromosome, i.e. without directed integration at a specific site.

The term “site-specific integration” as used herein refers to directed incorporation of a nucleic acid at a specifically chosen site of a chromosome. For example, site-specific integration can be achieved by homologous recombination or with the CRISPR/Cas9 system. Specific examples employ a site specific recombination system well known in the art. While Cre-lox recombination is the most widely used site-specific recombination system, other systems may be used such as the Flp-FRT recombination system, Dre-rox recombination system. PhiC31-attP/attB or another of the phage integrases.

The term “homologous recombination” as used herein refers to a gene targeting means for artificially modifying a specific gene on a chromosome or a genome. When a genomic fragment having a portion homologous to that of a target sequence on the chromosome is introduced into cells, the term refers to recombination that takes place based on the nucleotide sequence homology between the introduced genomic fragment and the locus corresponding thereto on the chromosome.

As used herein, “locus” refers to a specific location or DNA sequence on a chromosome. A locus can be characterized by endogeneous regulatory sequences which support expression of proteins.

Preferred loci are Rosa26, Hprt, b-actin and Rps21 or generally loci harboring housekeeping genes with high expression levels for site-specific integration. For random integration using artificial chromosomes such as BACs containing such loci or any other form of chromatin modifiers stabilizing open chromatin sites for gene expression, any site allowing the integration of the vector DNA into the host cell genome is suitable, particularly any euchromatin containing site.

The term “selection efficiency” refers to the number of desired cells that are selected based on predefined parameters out of a repertoire of cells. It is expressed as x selected cells (also referred to as “hits”) out of at least y number of cells in the repertoire. With a higher selection efficiency, a larger repertoire of cells can be screened to identify the best hits. The hits selected from the repertoire of transfected and/or recombinant host cells are particularly characterized by the high productivity for the respective protein-of-interest of the production host cell.

Using flow cytometry or similar systems for cell sorting, 10 million transfected cells can be analysed per hour and the best 100 cells from 10 million can be selected by this method and sorted into cell culture plates such as 96-well or 384-well plates. This includes, that also less than 10 million cells can be analysed, and just a single best cell can be sorted, or the cells sorted could be adjusted to the best 0.01%, or up to the best 0.1%, or best 1% or up to the best 10%. If more than 10 million transfected cells are available, then also up to 100 million cells or even more can be sorted, provided that the sorting procedure is not causing increased cell death thereby interfering with the selection criteria. An arbitrary number of cells can be collected by setting the limit to the percentage of best cells according to the numbers of cells, which can be handled for isolation and cultivation.

An arbitrary number can also be plated in limiting dilution form transfected cell pools. However, the selected cells from the pools are plated without further quality criteria. Therefore a large number of cells need to be plated and screened to obtain an increased probability for identified high producers. Typically, cells are seeded in 384 or 96 well plates and screened for their proliferation and production properties with more than 5 plates and frequently with robotic systems. There is additionally another drawback when plating the cells via limiting dilution, as there is just an average number of cells plated with a high degree of uncertainty, about the exact number plated. For example, when the cell concentration is adjusted to 10 cells per milliliter, and 100 μl per well is plated, then in average 1 cell is plated per well. This includes, that frequently according to statistics, two cells or no cell are found per well. Thus, to obtain single clones with high certainty, a second cell cloning step is required. Therefore, limiting dilution requires considerable time and human and material resources for obtaining high producing single clones.

In the examples described, 1 million cells were transfected each for generating pools and subsequent limiting dilutions or for fast generation of stable clones and sorting the best 96 clones via flow cytometry. Either a stable pool with prolonged antibiotic selection was generated and afterwards plated in 96 well plates via limiting dilution without any further selection criteria, or host cells were transfected and selected 1 or 2 days after transfection for a short period of time under high antibiotic concentrations followed by single cell sorting via flow cytometry to isolate the best 96 clones from 1 million transfectants, thereby achieving a selection efficiency of 1 clone out of at least 10⁴ cells.

Therefore, the present invention is based on a novel method for identifying and selecting single cells to generate stable and high-producer production cell lines. The method is basically employing single cell sorting of a repertoire of recombinant host cells based on intrinsic physical biomarkers. According to an example, a single cell clone for generating a stable production cell line can be isolated within one week after transfection. In particular, the single cell clone was identified from a pool of stably transfected cells by measuring basic cellular properties employing forward scatter (FSC) as an indicator of cell size and side scatter (SSC) as an indicator for granularity of a cell

The method as described herein provides several advantages over the existing techniques for isolation of production clones and generation of stable and efficient production cell lines:
1. Cuts back on time by at least 4 months (compared to a conventional method of using stable cell pools to perform limiting dilution serial dilutions and/or recloning of clones upon first selection)
2. Uses basic cellular properties such as cell size and granularity to differentiate between transfected and untransfected cells
3. When using an antibiotic resistance selection marker and a high antibiotic concentration during selection,
   a. any proliferation advantage during initial stages after transfection can be circumvented;
   b. due to limited proliferation in high antibiotics the variability between the isolated single clones are higher, as a result providing a better chance to isolate the “high producer”
   c. A linear correlation between copy numbers of recombinant DNA to protein production can be shown. Conversely, the survival of only those cells that have high integration events under the selection conditions is predicted.
   d. A generic pre-screening that utilizes antibiotic resistance as a tool to identify potential high producers is preferred.

The foregoing description will be more fully understood with reference to the following examples. Such examples are, however, merely representative of methods of practicing one or more embodiments of the present invention and should not be read as limiting the scope of invention.

### EXAMPLES

### Example 1

Generation of Single Clones Expressing Recombinant Intracellular Protein eGFP (Enhanced Green Fluorescent Protein)

Construction of a BAC-eGFP

For BAC-eGFP construction, 5 μg of the plasmid-eGFP DNA (Sequence IDXX, vector map in FIG. 10) was digested with fast digest restriction enzymes SfaAI (Thermo Fisher Scientific, cat. no. FD2094) and PacI (Thermo Fisher Scientific, cat. no. FD2204) (5U each) for 30 min at 37° C. The fragments were then resolved on a 1% Agrarose-TAE gel. The slower migrating fragment contained the gene-of-interest and the homology arms for BAC recombineering. This fragment was cut out of the gel and purified by Sigma Gel extraction kit (Sigma-Aldrich, part of Merck; NA1111-1K) according to manufacturer's instructions. The concentration of the DNA fragment was then measured using a UV spectrophotometer at 260 nm. 150 ng of the purified SfaAI/PacI fragment was electroporated into E. coli DH10b electrocompetent cells induced for recombination enzymes (material can be obtained from Gene Bridges GmbH, Heidelberg, procedures according to the pRed/ET manual by Gene Bridges) and containing the Rosa26BAC (can be obtained from the BACPAC Resources Center, Children's Hospital Oakland Research Institute (CHORI), Oakland, Calif., USA, clone name RP24-85L15), a BAC comprising the sequence of the Rosa26 locus (SEQ ID NO:1) using a Bio-Rad electroporator at 2000V/2 Ohms. The transformants were recovered for 70 min at 37° C. 100 μL of the transformation was plated on an LB-agar plate containing 12.5 μg/mL of Chloramphenicol (Sigma; C1919-5G) and 15 μg/mL of Kanamycin. The plates were then incubated overnight at 37° C. Positive colonies were picked for performing BAC DNA isolation in LB culture containing 12.5 μg/mL of Chloramphenicol and 15 μg/mL of Kanamycin. DNA isolation was done by spinning down the culture at 4000 rpm for 5 min. The cell pellet was resuspended in 300 μL of P1 buffer containing RNase A (Qiagen Miniprep kit; 12163) followed by 300 μL of P2 buffer. The tube was inverted 5 times gently at room temperature. Soon after, 300 μL of buffer P3 was added and inverted to mix 5 times and incubated on ice for 10 min. 600 μL of isopropanol were added and incubated at −20° C. for 20 min. The mixture was then spun down at 14000 rpm for 30 min at room temperature. The supernatant was carefully discarded without disturbing the pellet and the pellet washed once with 500 μL of 70% ethanol. The spinning was repeated at 14000 rpm for 15 min. The supernatant was discarded carefully without disturbing the pellet. The pellet was dried for 5 min and then solubilized in 30 μL of 10 mM Tris buffer [pH 8.0]. The integration of the linear fragment into the Rosa26 BAC was verified by digestion of the isolated DNA by EcoRI (Thermo Fisher Scientific; cat no. ER0271) for characteristic BAC fragmentation analysis. 20 μL of BAC DNA was digested with 1U of EcoRI for 30 min and resolved the products of the reaction on a 1% Agarose-TAE gel. Further, the integration was also verified by PCR analysis for (a) the 5′ homologous arm insertion site using a forward primer (AB11) that binds upstream of the integration site in the BAC and a reverse primer (AB12) that binds in the 5′ region of the incoming DNA fragment containing the gene-of-interest (in this case eGFP), (b) gene-of-interest primers that are specific to the eGFP fragment to ensure that the gene is present using forward primer (AB09) and reverse primer (AB40), and (c) the 3′ homologous arm insertion site using a forward primer (AB13) that binds in the 3′ region of the incoming DNA fragment and a reverse primer (AB14) that anneals to a region downstream of the integration site in the BAC. To isolate BAC DNA for transfection, a DH10b colony containing the confirmed modified Rosa26 BAC was inoculated into a 500 mL LB-medium containing 12.5 μg/mL Chloramphenicol and 15 μg/mL Kanamycin. The BAC DNA was then isolated using a NucleoBond Xtra BAC isolation kit (Macharey-Nagel; 740436.25) and the concentration was measured using a UV spectrophotometer at 260 nm. 6 μg of BAC DNA was linearized using 0.5U of PI-SceI enzyme (New England Biolabs; R0696L) to linearize the BAC overnight in a final volume of 10 μL.

Primers used for sequencing and/or PCR verification:

| Primer | Sequence | Primer description |
|---|---|---|
| AB09 | CAGGGGGACGGCTGCCTTCGG | Forward primer binds in CAGGS promoter |
| | SEQ ID NO: 7 | |
| AB10 | GCGAAGGAGCAAAGCTGCTATTG | Reverse primer binds in neomycin |
| | SEQ ID NO: 8 | |
| AB40 | GGTGGCATCGCCCTCGCCCTC | Reverse primer to screen by colony PCR |
| | SEQ ID NO: 9 | for integration and right orientation |
| | | ofeGFP fragment into pAB3 |
| AB11 | CCAACACAGATGAGCCTAAGCC | Forward primer to screen for |
| | SEQ ID NO: 10 | recombination at 5′ insertion site of BAC |
| AB12 | AACTAATGACCCCGTAATTGATTAC | Reverse primer to screen for |
| | SEQ ID NO: 11 | recombination at 5′ insertion site of BAC |
| AB13 | CATCGCCTTCTATCGCCTTCTTG | Forward primer to screen for |
| | SEQ ID NO: 12 | recombination at 3′ insertion site of BAC |
| AB14 | AACCTGAGCCAGACTTTCCACTGCAATATC | Reverse primer to screen for |
| | SEQ ID NO: 13 | recombination at 3′ insertion site of BAC |
| AB88 | GTGCGTGTTCACTCGACC | Reverse primer to screen by colony PCR |
| | SEQ ID NO: 14 | for integration and right orientation of |
| | | FGF23 (C-terminus) into base vector |

Transfection of Mammalian Cells

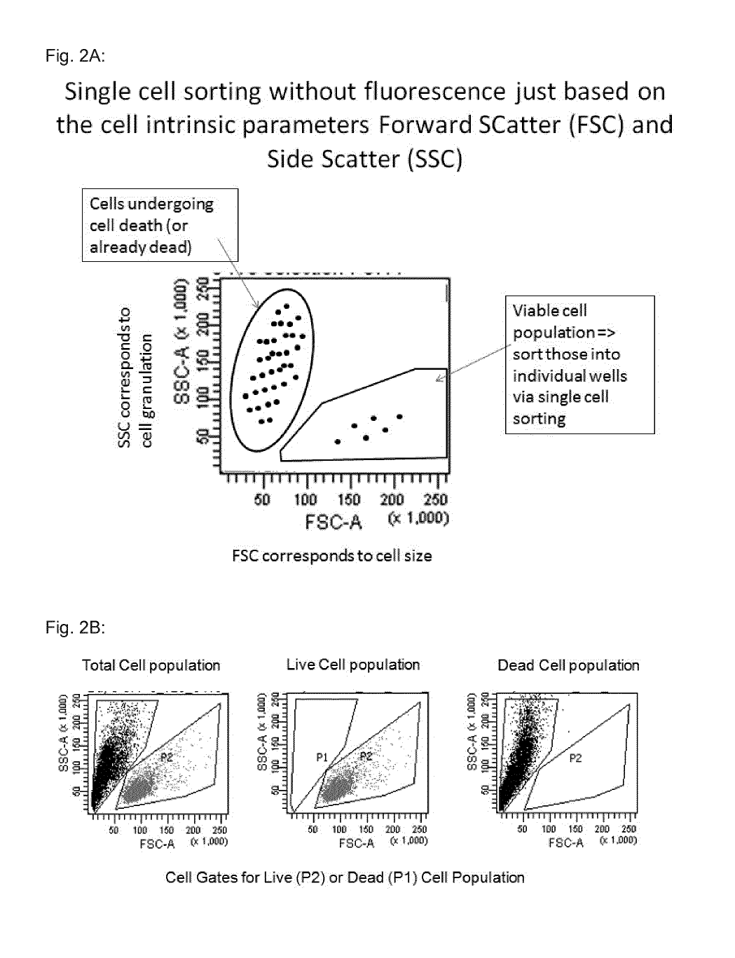

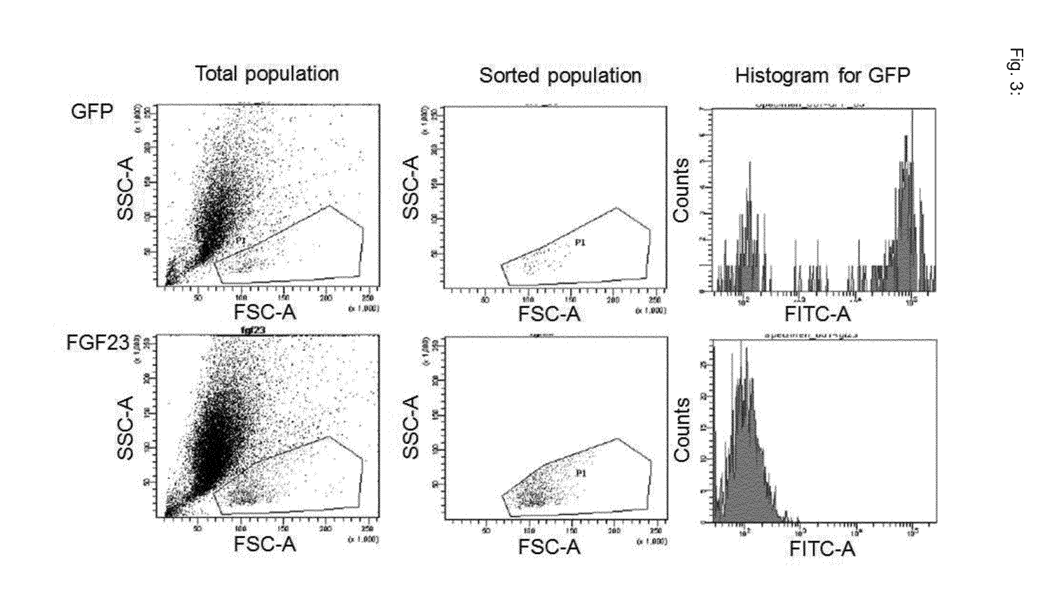

1×10⁶ cells were transfected with 5 μg of GFP-BAC DNA for intracellular GFP expression. Expression of GFP was used to establish the protocol and to follow the different stages during transfection. On day 2 after transfection, cells were cultivated in the presence of 0.25 mg/mL G418 (Roth) for 2 days. After 2 days, G418 concentration was increased to 0.5 mg/mL and kept in selection for 2 more days. On day 4 after antibiotic selection started, the culture was split to two halves—for one half, G418 was retained at 0.5 mg/mL while for the other half the G418 concentration was increased to 1.0 mg/mL. Aliquots of the cells were analyzed periodically during antibiotics treatment by FACS analysis using Propidium Iodine staining as a marker for dead cells until the following criteria were met in order to decide when single cells were to be sorted into 96-well plates:
majority of host cell population shows signs of cell death due to toxicity with high antibiotica concentrations
small viable subpopulation (<5% of the total) of transfected cells are resistant under similar conditions
differences in FSC-SSC characteristics for live and dead population are clearly visible (FIG. 2)

10 days after transfection, i.e. after cultivating and selecting the transfected cells in the presence of G418, cells were prepared for sorting by passing them through 100 μm cell strainer to remove any clumps, and sorted solely based on FSC and SSC by the flow cytometer FACS Aria III from Becton Dickinson with a Voltage setting of 140V for FSC-A and 250V for SSC-A. In a FSC/SSC plot (FSC-A on the x-axis, SSC-A on the y-axis), the asymmetric live gate is between 60 and 250 units in the FSC, and between 10 and 150 units in the SSC, starting narrow on the left bottom side and getting broader to the right and upper side (FIG. 3 upper panel). Although GFP expression was not used as a criterium for sorting, the GFP expression was recorded in the green fluorescent channel for the sorted live cells (FIG. 3, Histogram). The single cells were sorted into medium containing 96 well plates in the absence of lethal antibiotics concentrations. The best 96 cells out of 10⁶ cells transfected were sorted to result in a selection efficiency of about 1 in 10⁴. Single cells were expanded appropriately first in 96-well round bottom plate containing 50 μL of CD-CHO media supplemented with 1 mM Glutamine (Lonza), 0.2% Anti-clumping reagent (Invitrogen) and 0.001% Phenol Red (Sigma). After about 17 divisions, the cells were in sufficient number to characterize the clone, analyze for protein production and prepare freezer stocks.

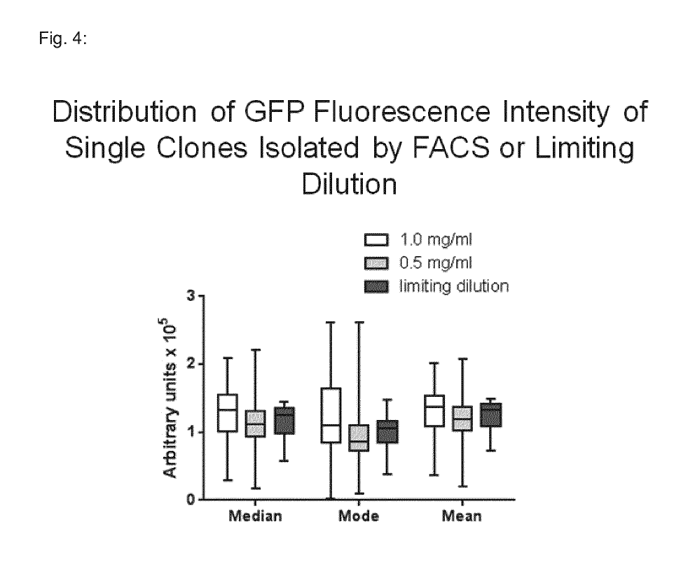

After about 10 cell divisions (equates to 1024 cells), the individual clones were resuspended and transferred to 24-well plates containing 500 μL of supplemented CD-CHO medium. Following another 5 divisions, cells were analysed for their GFP expression by FACS analysis in the presence of PI as a marker for dead cells. For each clone, the GFP fluorescence intensity parameters, such as mean, median and mode were quantified and a box-and-whisker plot was created for analysis of the respective statistical parameters (FIG. 4). The result indicates that among the clones sorted according to our described method by flow cytometry, individual clones with higher expression levels (25% best clones) were selected, and these clones were not found among those generated by limiting dilutions. Thus, for this best 25% of production cells the selection efficiency was 2.5 cells per 10⁵ transfectants.

In a comparative example, it would be necessary to screen more than 100 clones with conventional techniques such as limiting dilution to identify such high producer clone (if any is generated or left from the cell pools) there.

In the present experiment shown, such high producer clones were not found via limiting dilutions, but with direct single cell sorting. Additionally, the average value for fluorescence intensity of the selected clones was increased with increasing G418 concentration during the early selection phase as can be seen by the comparison between the average value of the clones selected in 0.5 mg/ml G418 and 1.0 mg/ml, respectively.

### Example 2

Construction of a BAC with a FGF23 Expression Cassette for Secreted Expression of C-Terminal Fragment of FGF23

For construction of the FGF23-BAC, a vector containing all the necessary genetic elements in addition to the coding sequence of the C-terminal fragment of human FGF23 was used (FIG. 10B, SEQ ID NO:15) In short, the FGF23 gene was placed under control of the chicken beta-actin gene promoter followed by a poly-adenylation signal. The cassette contains a neomycin/kanamycin resistance gene. The cassette is framed by 3′- and 5′-homology sequences for recombination into the bacterial artificial chromosome containing the ROSA 26 locus.

For BAC-FGF23 construction, from the plasmid construct as described above, 5 μg of DNA was digested with fast digest restriction enzymes SfaAI (Thermo Fisher Scientific, cat. no. FD2094) and PacI (Thermo Fisher Scientific, cat. no. FD2204) (5U each) for 30 min at 37° C. The fragments were then resolved on a 1% Agrarose-TAE gel. The slower migrating fragment contained the gene-of-interest and the homology arms for BAC recombineering. This fragment was cut out of the gel and purified by Sigma Gel extraction kit (Sigma-Aldrich, part of Merck; NA1111-1K) according to manufacturer's instructions. The concentration of the DNA fragment was then measured using a UV spectrophotometer at 260 nm. 150 ng of the purified SfaAI/PacI fragment was electroporated into E. coli DH10b electrocompetent cells induced for recombination enzymes (material can be obtained from Gene Bridges GmbH, Heidelberg, procedures according to the pRed/ET manual by Gene Bridges) and containing the Rosa26BAC (the Rosa26BAC can be obtained from the BACPAC Resources Center, Children's Hospital Oakland Research Institute (CHORI), Oakland, Calif., USA, clone name RP24-85L15) a BAC comprising the sequence of the Rosa26 locus, SEQ ID NO:1) using a Bio-Rad electroporator at 2000V/2 Ohms. The transformants were recovered for 70 min at 37° C. 100 μL of the transformation was plated on an LB-agar plate containing 12.5 μg/mL of Chloramphenicol (Sigma; C1919-5G) and 15 μg/mL of Kanamycin. The plates were then incubated overnight at 37° C. Positive colonies were picked for performing BAC DNA isolation in LB culture containing 12.5 μg/mL of Chloramphenicol and 15 μg/mL of Kanamycin. DNA isolation was done by spinning down the culture at 4000 rpm for 5 min. The cell pellet was resuspended in 300 μL of P1 buffer containing RNase A (Qiagen Miniprep kit; 12163) followed by 300 μL of P2 buffer. The tube was inverted 5 times gently at room temperature. Soon after, 300 μL of buffer P3 was added and inverted to mix 5 times and incubated on ice for 10 min. 600 μL of isopropanol were added and incubated at −20° C. for 20 min. The mixture was then spun down at 14000 rpm for 30 min at room temperature. The supernatant was carefully discarded without disturbing the pellet and the pellet washed once with 500 μL of 70% ethanol. The spinning was repeated at 14000 rpm for 15 min. The supernatant was discarded carefully without disturbing the pellet. The pellet was dried for 5 min and then solubilized in 30 μL of 10 mM Tris buffer [pH 8.0]. The integration of the linear fragment into the Rosa26 BAC was verified by digestion of the isolated DNA by EcoRI (Thermo Fisher Scientific; cat no. ER0271) for characteristic BAC fragmentation analysis. 20 μL of BAC DNA was digested with 1U of EcoRI for 30 min and resolved the products of the reaction on a 1% Agarose-TAE gel. Further, the integration was also verified by PCR analysis for (a) the 5′ homologous arm insertion site using a forward primer (AB11) that binds upstream of the integration site in the BAC and a reverse primer (AB12) that binds in the 5′ region of the incoming DNA fragment containing the gene-of-interest (in this case FGF23), (b) gene-of-interest primers that are specific to the FGF23 fragment to ensure that the gene is present using forward primer (AB09) and reverse primer (AB88), and (c) the 3′ homologous arm insertion site using a forward primer (AB13) that binds in the 3′ region of the incoming DNA fragment and a reverse primer (AB14) that anneals to a region downstream of the integration site in the BAC. To isolate BAC DNA for transfection, a DH10b colony containing the confirmed modified Rosa26 BAC was inoculated into a 500 mL LB-medium containing 12.5 μg/mL Chloramphenicol and 15 μg/mL Kanamycin. The BAC DNA was then isolated using a NucleoBond Xtra BAC isolation kit (Macharey-Nagel; 740436.25) and the concentration was measured using a UV spectrophotometer at 260 nm. 6 μg of BAC DNA was linearized using 0.5U of PI-SceI enzyme (New England Biolabs; R0696L) to linearize the BAC overnight in a final volume of 10 μL.

Transfection into Mammalian Cells

1×10⁶ cells were transfected with 5 μg of FGF23-BAC DNA for expression of secreted FGF23. On day 2 after transfection, cells were cultivated in the presence of 0.25 mg/mL G418 (Roth) for 2 days. After 2 days, G418 concentration was increased to 0.5 mg/mL and kept in selection for 2 more days. On day 4 after antibiotic selection started, the culture was split to two halves—for one half, G418 was retained at 0.5 mg/mL while for the other half the G418 concentration was increased to 1.0 mg/mL. 10 days after transfection during which the transfected cells were cultivated in the presence of G418, cells were prepared for sorting by passing them through 100 μm cell strainer to remove any clumps, and sorted solely based on FSC and SSC by the flow cytometer FACS Aria III from Becton Dickinson with a Voltage setting of 140V for FSC-A and 250V for SSC-A. In a FSC/SSC plot (FSC-A on the x-axis, SSC-A on the y-axis), the asymmetric live gate is between 60 and 250 units in the FSC, and between 10 and 150 units in the SSC, starting narrow on the left bottom side and getting broader to the right and upper side (FIG. 3 lower panel). The single cells were sorted into medium containing 96 well plates in the absence of lethal antibiotics concentrations. The selection efficiency in this example was again 96 cells out of 10⁶ total transfectants, resulting in about 1 out of 10⁴ cells, Single cells were expanded appropriately first in 96-well round bottom plate containing 50 μL of CD-CHO media supplemented with 1 mM Glutamine (Lonza), 0.2% Anti-clumping reagent (Invitrogen) and 0.001% Phenol Red (Sigma). After about 17 divisions, the cells were in sufficient number to characterize the clone, analyze for protein production and prepare freezer stocks.

After about 10 cell divisions (equates to 1024 cells), the individual clones were resuspended and transferred to 24-well plates containing 500 μL of supplemented CD-CHO medium.

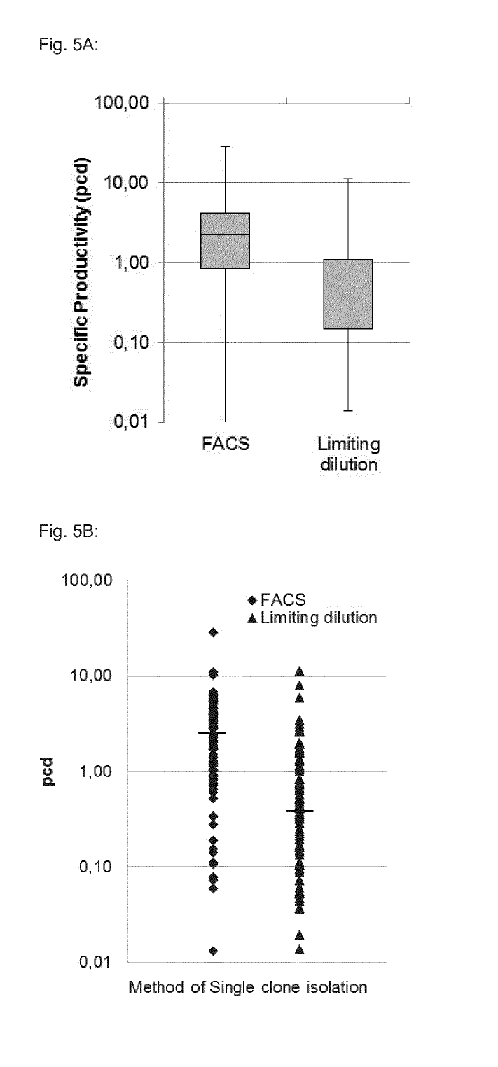

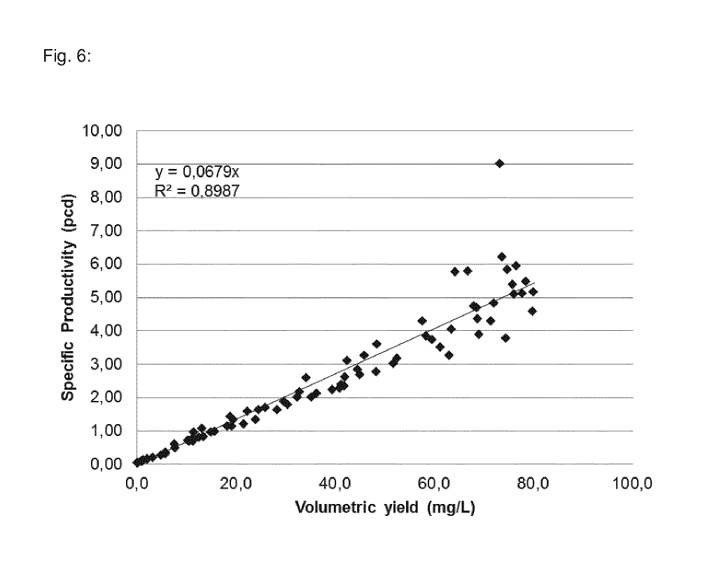

Single clones were analyzed for production under fed-batch conditions in 96-well plates. For production, cells were seeded in 96-well plates at 1×10⁵ cells/well in 100 μL of production medium (supplemented CD-CHO described above was mixed with 15% Feed B CD-CHO (Invitrogen) and 3.3% Function^{MAX} titer enhancer (Invitrogen)). The plates were incubated without shaking. Feed supplement was added to culture every 2 days (Feed B CD-CHO at a concentration of 10% culture volume and Function^{MAX} titer enhancer at a concentration of 3.3% culture volume). Cultures were spun down at the end of 8-days and collected the supernatants for analysis of secreted proteins by ELISA. As with GFP analysis, a similar setup was performed for FGF23 by limiting dilution for comparison. Specific productivity for both methods was analyzed by an FGF23 ELISA (Biomedica, Austria) according to the manufacturer's instructions. The pcd values for the individual clones of the respective group were statistically analysed and plotted by a box-and-whisker plot and scatter plot, respectively (FIGS. 5A and 5B). The volumetric yield for each clone was calculated and the correlation between pcd values and volumetric yields of these clones were analysed (FIG. 6). The results show that the mean value for specific productivity of clones sorted by flow cytometry was about 10 times (1 log) higher than the mean value of those clones sorted by limiting dilution. Again this demonstrates that the screening efficiency to identify high producers is strongly improved.

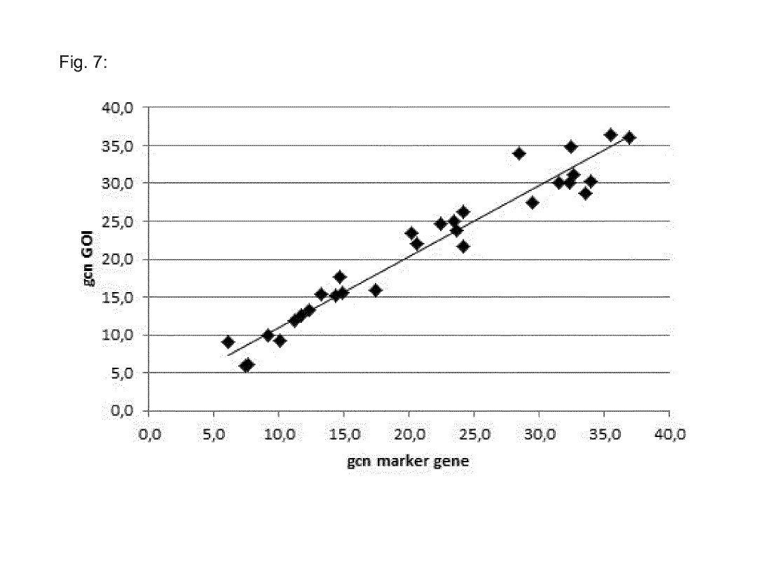

The gene copy number for the GOI for the individual clones correlates well with the specific productivity of the POI. Thus, the correlation between the gene copy number of the GOI and the gene copy number of the marker gene is of interest. This can be tested using real time PCR with specific primers for the respective gene. The results from RT-PCR show a correlation between these two genes according to FIG. 7.

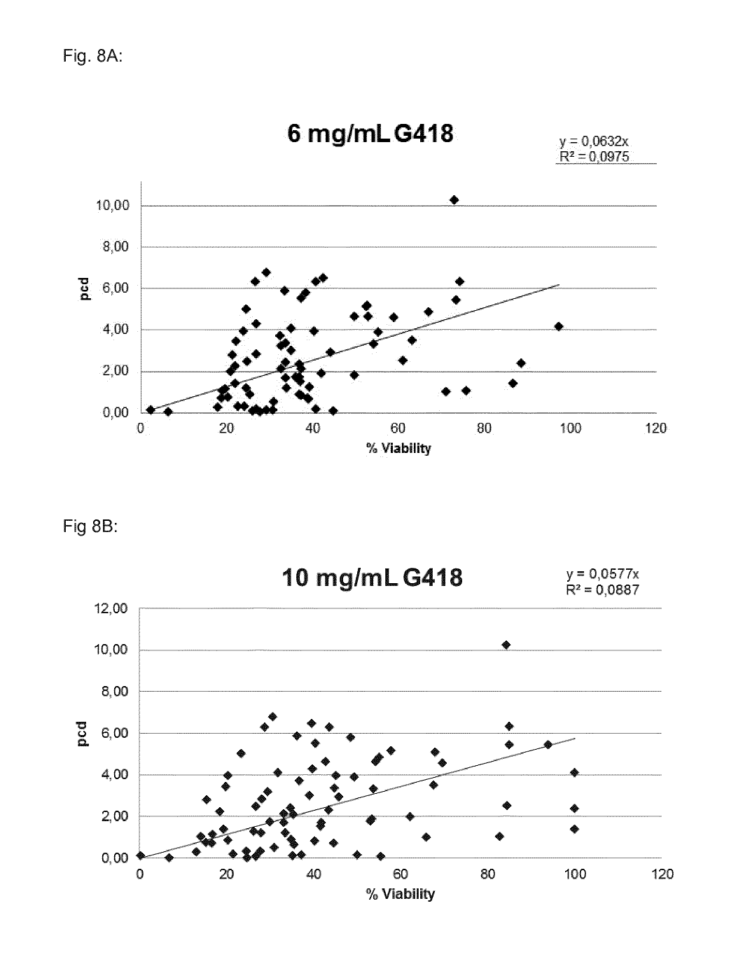

In order to test the functional correlation between the POI production and the marker gene function, selected clones producing recombinant FGF23 with determined pcd values were analysed for their survival under high antibiotic concentration. For this, 1×10⁵ of cells/well were seeded in 100 μL of CD-CHO medium (supplemented with L-glutamine and anti-clumping reagent) in 96-well plates. Cells were treated with 6 mg/mL or 10 mg/mL of G418 for 3 days. As controls, the cells were cultivated in a similar setup without antibiotics. Cell viability was measured using Abcam Cell Cytotoxicity assay kit as per manufacturer's instructions. 20 μL of cell cytotoxicity reagent was added to each well and incubated for 3 h at 37° C. An increase in absorbance at 570 nm coupled with a simultaneous decrease in absorbance at 605 nm indicates the presence of live cells. A ratio of live cell population observed in antibiotic-treated samples to untreated controls for each clone provides an insight into how much antibiotic a cell can tolerate (FIG. 8). A correlation between increased productivity and resistance to high antibiotic concentration was observed. The data demonstrate that a generic screening method based on resistance to high antibiotic concentrations can be used to pre-screen the large sample size to a relatively small number for further testing.

### Example 3

Identifying Early Timepoints for the Generation of Single Clones Expressing Recombinant Intracellular Protein

Several aliquots of 1×10⁵ or 1×10⁶ cells were each transfected with 5 μg or 25 μg of GFP-Rosa26-BAC DNA (either circular or linearized in the BAC backbone with SceI) for intracellular GFP expression using Amaxa Nucleofector kit. Expression of GFP was used to evaluate protocols for improving transfection and selection conditions and to follow the different stages during transfection. On day 1 after transfection, 1.0 mg/ml G418 (Roth) was added to the culture medium and cells were continued to be cultivated in the presence of 1.0 mg/mL G418. Aliquots of the cells were monitored from day 3 until day 9 post-transfection by FACS analysis, and beside the Forward Scatter and Side Scatter characteristics, Propidium Iodine staining was used as a marker for dead cells.

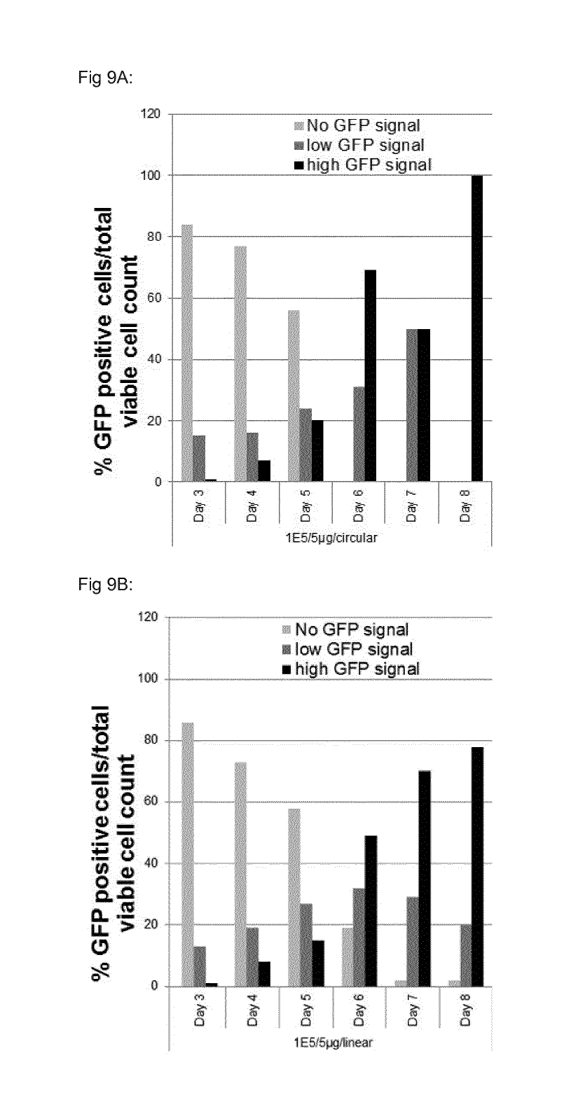

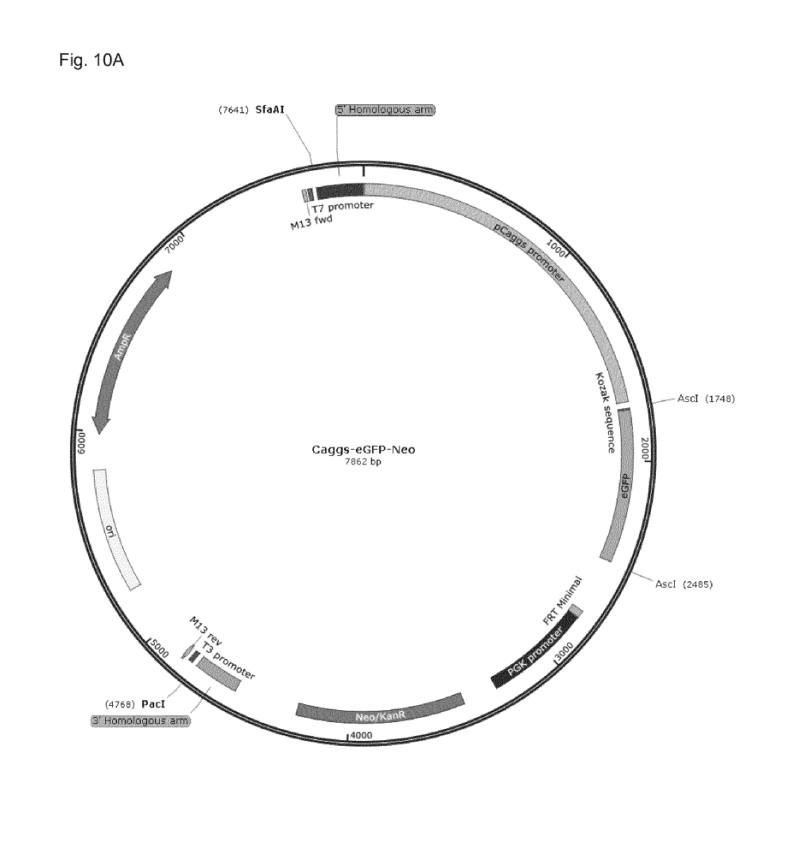

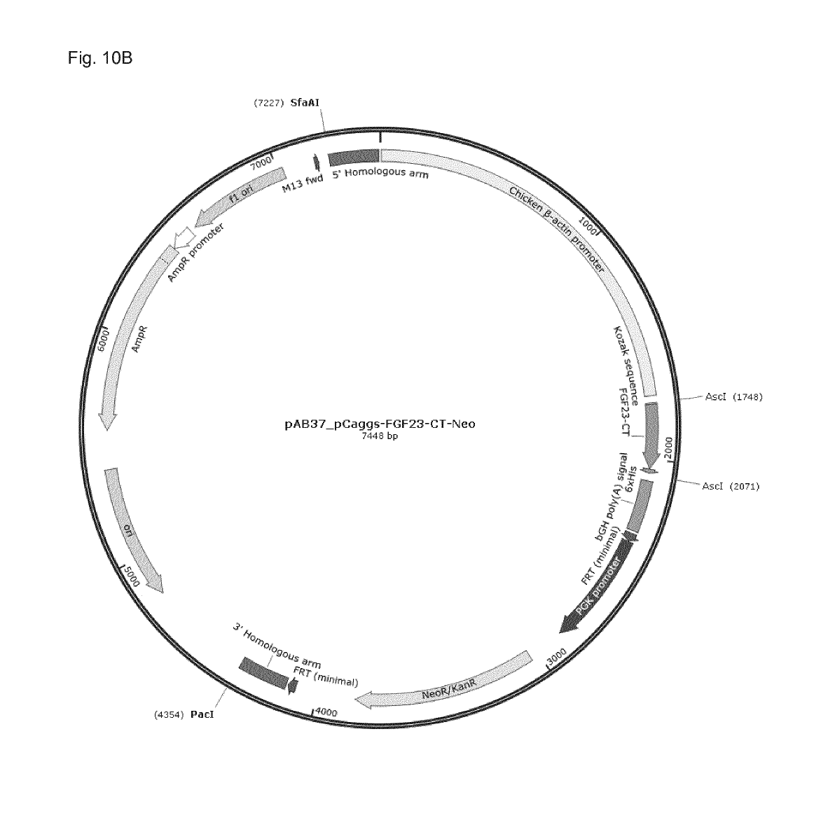

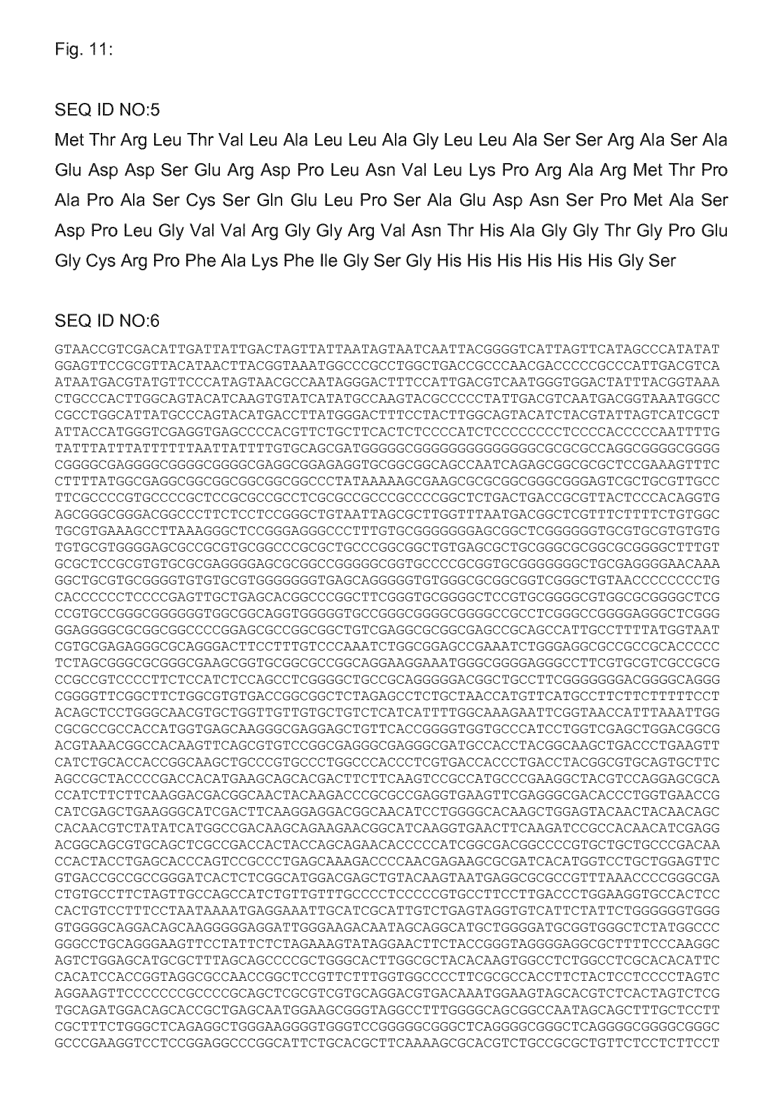

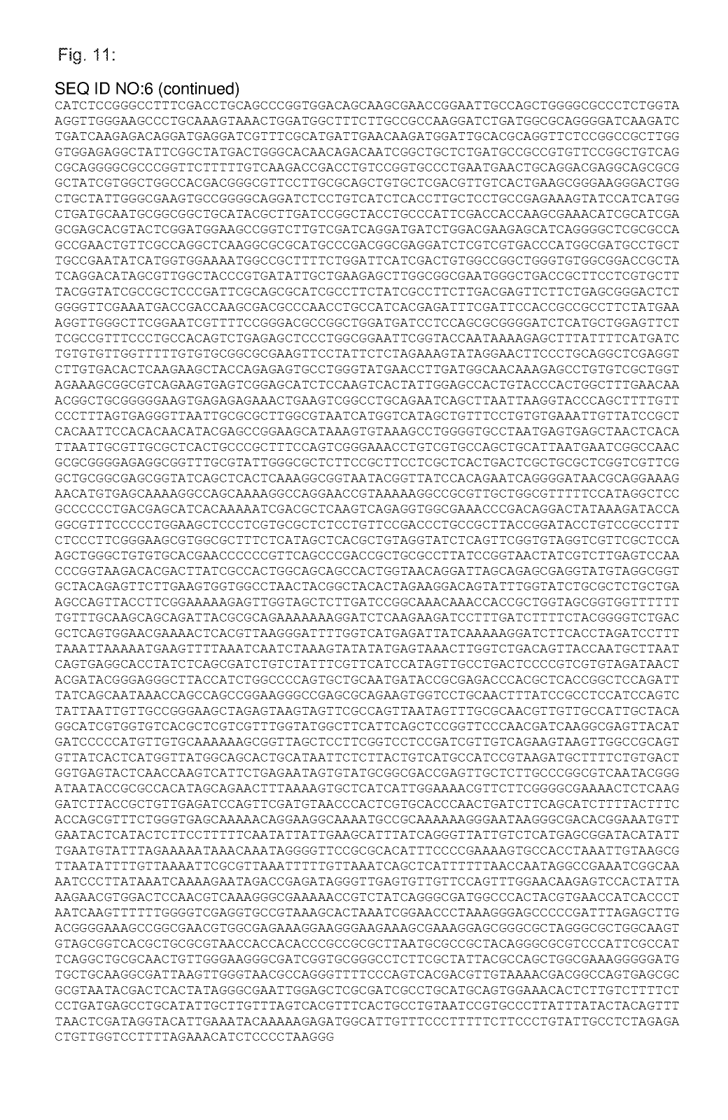

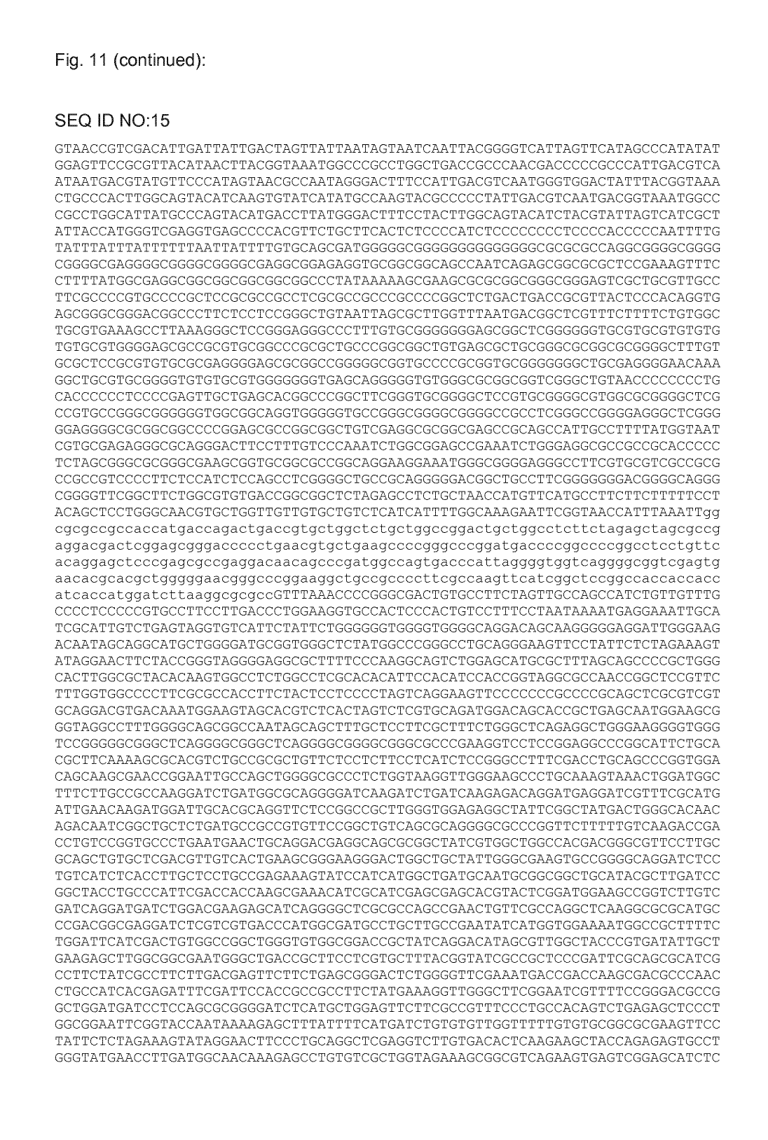

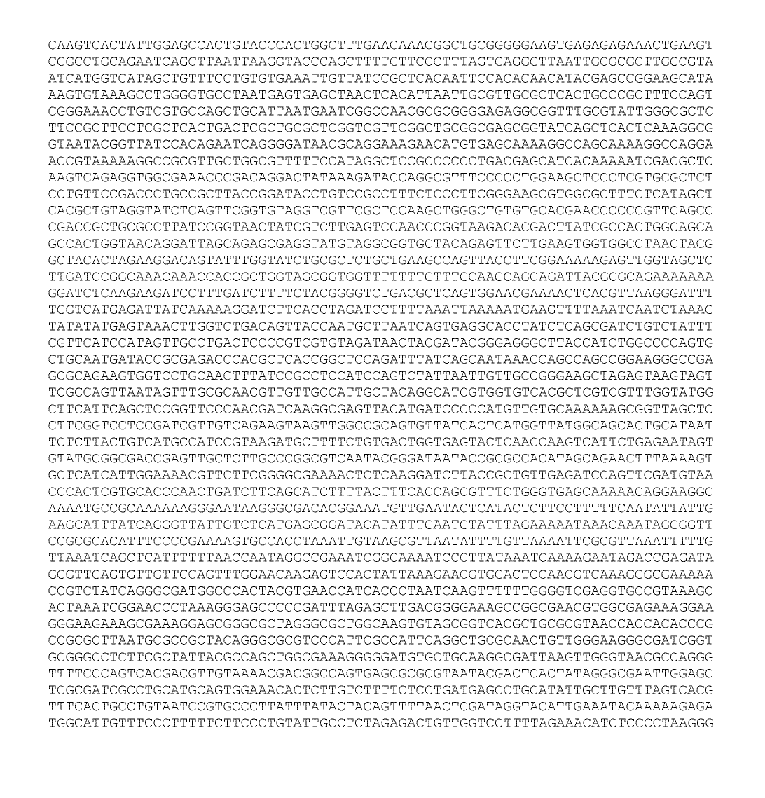

Only live cell population was gated and the gated cells were further divided into 3 categories—no GFP expression (<100 arbitrary units of fluorescence signal intensity) equivalent to the negative control of CHO cells without GFP expression, low GFP expression (between 100-10,000 arbitrary units of fluorescence signal intensity) and high GFP expression (>10,000 arbitrary units of fluorescence signal intensity). GFP signal intensity for each category above was monitored from day 3 to day 9 and % for each category was calculated by dividing the number of cells within the category by the sum of the cell numbers within all three categories. Comparison of cell-to-DNA ratio showed that 5 or 25 μg of DNA can be used for transfection, and the cell number can vary between 1×10⁵ to 1×10⁶ cells. Using 5 μg Rosa26-BAC DNA for 1×10⁵ cells showed in this experiment better transfection efficiency than using 25 μg DNA for 1×10⁶ cells. When 1×10⁵ cells were transfected with either 5 μg of linear or circular DNA and selected from day 1 on after transfection with 1.0 mg/ml G418, 6-9 days after transfection (which corresponds to 5-8 days after start of the selection) were observed as good time points for flow cytometry sorting of the remaining viable cells (FIG. 9A for transfection with the circular BAC and FIG. 9B for transfection with the linear BAC). From day 6 post transfection on, at least 50% of the viable cells belonged to the high expressing cells. This fraction of high expressing cells in the viable cell population was increasing to about 80% for the linearized BAC, and to 100% for the circular BAC. In the case of the circular BAC, this means that from day 6-8, 1 to 3 cells out of 10⁴ cells are the cells of interest, showing high expression for our protein of interest (Table 1). For the linearized BACs, 427-332 cells out of 10⁴ cells are the cells of interest, showing high expression for our protein of interest.

TABLE 1
Cell counts obtain in the various gates from the
transfected cells as described in example 3 and in FIG. 9.

| | | GFP intensity | | | | |
|---|---|---|---|---|---|---|
| | | no | low | high | VCC | TE |
| 1E5/5 μg/circular | Day 3 | 3050 | 448 | 143 | 3641 | 10000 |
| | Day 4 | 296 | 61 | 25 | 382 | 7035 |
| | Day 5 | 85 | 36 | 30 | 151 | 10000 |
| | Day 6 | 0 | 5 | 11 | 16 | 10000 |
| | Day 7 | 0 | 1 | 1 | 2 | 5845 |
| | Day 8 | 0 | 0 | 1 | 1 | 10000 |
| 1E5/5 μg/linear | Day 3 | 1119 | 302 | 59 | 1480 | 5250 |
| | Day 4 | 1486 | 379 | 172 | 2037 | 10000 |
| | Day 5 | 655 | 308 | 166 | 1129 | 10000 |
| | Day 6 | 83 | 136 | 208 | 427 | 10000 |
| | Day 7 | 5 | 96 | 231 | 332 | 10000 |
| | Day 8 | 8 | 74 | 284 | 366 | 10000 |

GFP intensity definition used: “no”: less than 100 arbitrary units of fluorescence signal intensity “low”: between 100-10,000 arbitrary units of fluorescence signal intensity “high”: more than 10,000 arbitrary units of fluorescence signal intensity
VCC: viable cell count
TE: total events

Material and Methods:

Transfection of host cell lines (Nucleofection): Mammalian Host cells, specifically CHO-K1, were cultured in appropriate commercial cell culture media (CD-CHO; Invitrogen) until the day of transfection. On the day of transfection, logarithmically growing cells were counted and 1×10⁶ cells were resuspended in 100 μL of Amaxa Nucleoporation buffer (Lonza). Resuspended cells were transferred to a nucleoporation cuvette (provided with kit). The sequence for GFP or FGF23 (SEQ ID NO:5) was introduced into plasmid or a BAC vector comprising locus Rosa26 (SEQ ID NO:1), see Zboray et al. 5 μg or 25 μg of plasmid DNA or BAC-DNA was pipetted into the electroporation cuvette containing the cells and the cells were electroporated according to the manufacturer's protocol. Transfected cells were immediately transferred to a 6-well plate containing 2 mL of fresh prewarmed medium. Antibiotica were added at lethal concentrations 1 or 2 days post-transfection.

Transfection of Host Cell line (Lipofection): Mammalian host cells, specifically CHO-K1, were cultured in appropriate culture media (CD-CHO; Invitrogen) until the day of transfection. 15 μL of Lipofectin (Invitrogen) was incubated with 5 μg of DNA for 30 min at room temperature for complexation. The lipofectin-DNA complex was then slowly overlaid on to 4×10⁵ CHO-K1 cells in a 6-well plate containing 2.5 mL of CD-CHO medium. All steps were followed according to Manufacturer's instructions. Cells were cultivated and allowed to recover for 1 or 2 days at 37° C. before the addition of lethal antibiotica concentrations.

Limiting dilution for production clone isolation: For limiting dilution of production clones out of cell pools, 4×10⁵ cells were transfected with lipofectin/5 μg BAC DNA as described above. The selection was done starting with 0.25 mg/ml G418 (Roth) 2 days post-transfection and gradually increasing to 0.75 mg/ml. Stable pools were generated within 16 days post-transfection. Cells were diluted to 0.5 cells/well and seeded in a 96-well round-bottom plate containing 100 μL of CD-CHO supplemented with L-Gln, phenol red, anti-clumping reagent and 0.1 mg/mL G418. Cells were expanded as mentioned earlier and analyzed for specific productivity (pcd) in case of secreted proteins or fluorescence intensity in case of intracellular expression of green fluorescent protein.

### Example 4

Comparison of a Conventional Plasmid and a BAC for Recombinant Protein Expression in Individual Mammalian Cells of a Cell Population and Cell Pools Respectively Early After Transfection and After Prolonged Culture

a) Plasmid-eGFP

A plasmid able to express eGFP in mammalian cells was constructed. The plasmid comprises the eGFP sequence driven by a the Caggs-promoter and an optimized Kozak-sequence just upstream of the eGFP start codon. The vector map is shown in FIG. 10.

b) BAC-eGFP Construction

For BAC-eGFP construction, from the plasmid-eGFP construct as described above, 5 μg of DNA was digested with fast digest restriction enzymes SfaAI (Thermo Fisher Scientific, cat. no. FD2094) and PacI (Thermo Fisher Scientific, cat. no. FD2204) (5U each) for 30 min at 37° C. The fragments were then resolved on a 1% Agrarose-TAE gel. The slower migrating fragment contained the gene-of-interest and the homology arms for BAC recombineering. This fragment was cut out of the gel and purified by Sigma Gel extraction kit (Sigma-Aldrich, part of Merck; NA1111-1K) according to manufacturer's instructions. The concentration of the DNA fragment was then measured using a UV spectrophotometer at 260 nm. 150 ng of the purified SfaAI/PacI fragment was electroporated into E. coli DH10b electrocompetent cells induced for recombination enzymes (material can be obtained from Gene Bridges GmbH, Heidelberg, procedures according to the pRed/ET manual by Gene Bridges) and containing the Rosa26BAC (can be obtained from the BACPAC Resources Center, Children's Hospital Oakland Research Institute (CHORI), Oakland, Calif., USA, clone name RP24-85L15), a BAC comprising the sequence of the Rosa26 locus (SEQ ID NO:1) using a Bio-Rad electroporator at 2000V/2 Ohms. The transformants were recovered for 70 min at 37° C. 100 μL of the transformation was plated on an LB-agar plate containing 12.5 μg/mL of Chloramphenicol (Sigma; C1919-5G) and 15 μg/mL of Kanamycin. The plates were then incubated overnight at 37° C. Positive colonies were picked for performing BAC DNA isolation in LB culture containing 12.5 μg/mL of Chloramphenicol and 15 μg/mL of Kanamycin. DNA isolation was done by spinning down the culture at 4000 rpm for 5 min. The cell pellet was resuspended in 300 μL of P1 buffer containing RNase A (Qiagen Miniprep kit; 12163) followed by 300 μL of P2 buffer. The tube was inverted 5 times gently at room temperature. Soon after, 300 μL of buffer P3 was added and inverted to mix 5 times and incubated on ice for 10 min. 600 μL of isopropanol were added and incubated at −20° C. for 20 min. The mixture was then spun down at 14000 rpm for 30 min at room temperature. The supernatant was carefully discarded without disturbing the pellet and the pellet washed once with 500 μL of 70% ethanol. The spinning was repeated at 14000 rpm for 15 min. The supernatant was discarded carefully without disturbing the pellet. The pellet was dried for 5 min and then solubilized in 30 μL of 10 mM Tris buffer [pH 8.0]. The integration of the linear fragment into the Rosa26 BAC was verified by digestion of the isolated DNA by EcoRI (Thermo Fisher Scientific; cat no. ER0271) for characteristic BAC fragmentation analysis. 20 μL of BAC DNA was digested with 1U of EcoRI for 30 min and resolved the products of the reaction on a 1% Agarose-TAE gel. Further, the integration was also verified by PCR analysis for (a) the 5′ homologous arm insertion site using a forward primer (AB11) that binds upstream of the integration site in the BAC and a reverse primer (AB12) that binds in the 5′ region of the incoming DNA fragment containing the gene-of-interest (in this case eGFP), (b) gene-of-interest primers that are specific to the eGFP fragment to ensure that the gene is present using forward primer (AB09) and reverse primer (AB40), and (c) the 3′ homologous arm insertion site using a forward primer (AB13) that binds in the 3′ region of the incoming DNA fragment and a reverse primer (AB14) that anneals to a region downstream of the integration site in the BAC. To isolate BAC DNA for transfection, a DH10b colony containing the confirmed modified Rosa26 BAC was inoculated into a 500 mL LB-medium containing 12.5 μg/mL Chloramphenicol and 15 μg/mL Kanamycin. The BAC DNA was then isolated using a NucleoBond Xtra BAC isolation kit (Macharey-Nagel; 740436.25) and the concentration was measured using a UV spectrophotometer at 260 nm. 6 μg of BAC DNA was linearized using 0.5U of PI-SceI enzyme (New England Biolabs; R0696L) to linearize the BAC overnight in a final volume of 10 μL.

c) Transfection into Mammalian Cells

600,000 CHO K1 cells (CHO-K1-AC-free, from Sigma-Aldrich, cat. no. 13080801) were transfected with either 5 μg of plasmid-eGFP or BAC-eGFP also containing a G418 selection marker as previously described (Zboray et al., 2015):

Transfection of linearized BAC-eGFP plasmid and plasmid-eGFP respectively was performed in CHO-K1 cells using Amaxa Nucleofector V kit (Lonza; VCA1003). Cells in the growth phase were first counted using a CASY counter. 600,000 cells were spun down at 1200 rpm for 5 min. The supernatants were discarded and the cells were resuspended in 100 μL of nucleofection kit V containing supplement 1. 8.5 μL of the linearized BAC-eGFP or of the eGFP-plasmid were added to the resuspended cells and mixed gently by flicking the tube. The contents were then transferred to a Nucleofection cuvette and nucleoporated using program U-023. Immediately after nucleofection, 500 μL of pre-warmed stock CD-CHO medium was added to the cells and transferred using a Pasteur pipet (provided by the Manufacturer) to a 6-well corning plate containing 1.5 ml CD-CHO medium. The stock CD-CHO medium had been prepared by mixing 1 L of Chemically-defined CHO medium (Thermo; 10743-029), 40 mL of 100 mM ultraglutamine (Lonza, BE17-605E/U1), 2 mL of anti-clumping agent (Gibco, 01-0057DG) and 2 mL of phenol red (Sigma, P0290).

d) Determination of Expression

On day 2 after transfection (day 2 p.t.), the cultures were analyzed via FACS (FACSCANTO II, BD) to follow eGFP expression and split into two aliquots.

Cell pools analysis (aliquot 1): from day 2 p.t. on 0.75 mg/mL of G418 was added to the culture. Viability and eGFP expression was recorded at day 9 p.t. by FACS after transfection and at day 21 after transfection (day 21 p.t.). The percentage of eGFP positive cells as well as the MFI (mean fluorescence intensity) of the eGFP positive cells were determined.

Cell clone analysis (aliquot 2): the transfected cells were prepared for sorting by passing them through 100 μm cell strainers to remove any clumps and were sorted on a FACS ARIA III based on eGFP expression of cells in the live-gate (by FSC/SSC) by setting the lower limit of the fluorescent gate at the arbitrary fluorescent units 10000. The live-gate on the FACS ARIA III with a Voltage setting of 140V for FSC-A and 250V for SSC-A was set asymmetrically with FSC between 60 and 250 units, and SSC between 10 and 150 units, starting narrow on the left bottom side and getting broader to the right and upper side. 96 cells of each pool were sorted into medium containing single wells of a 96 well plate in the absence of antibiotics. Single cells were expanded without any antibiotics selection appropriately first in 96-well plate containing 100 μL of CD-CHO media containing above-mentioned supplements and then transferred into 24 well plates containing 500 μl of the same medium. On day 21 after transfection (day 21 p.t.), those clones, which recovered and could be expanded, were again analyzed for eGFP expression via FACS. The clones with an MFI smaller than 6000 were grouped as no or low eGFP expression, those with an MFI between 6000 and 60000 were intermediate eGFP expressors, and those with and MFI higher than 60000 were high eGFP expressors.

### e) Results and Conclusions

Analysis of pools of cells transfected with an expression cassette on either a conventional plasmid or on a BAC with a large euchromatin locus, respectively:

Table 2 shows a comparison of pools of transfected cells, transfected either with an eGFP-expression cassette on a conventional plasmid or with an eGFP-expression cassette within the Rosa26 locus, an exogenous euchromatin locus on a BAC, respectively. The pools are cultivated under antibiotic selection pressure. The antibiotic resistance gene marker is provided along with the eGFP expression cassette.

2 days after transfection (day 2 p.t.) the percentage of cells positive for eGFP is lower in the BAC transfected culture as compared to the plasmid-transfected culture (0.5% vs 2%), however, both transfected cell pools show similar fluorescence (MFI around 6,200 and 6,600 respectively). This is already an indication that the BAC-transfected cells have a higher specific expression of eGFP as compared to the plasmid-transfected cells in the pool.

9 days after transfection the viability in both cell pools is similar (3% vs. 4%), however, the number of clones expressing eGFP is much higher with the plasmid-transfection (20%) as compared to the BAC-transfection (3.3%). This is an indication that with the conventional plasmid transfections, most of the eGFP-positive cells have already died due to the selection pressure.

The culture transfected with the BAC-eGFP show a similar number of living cells (4%) and eGFP-positive cells (3.3%), indicating that all cells that produce eGFP are alive. Moreover, the mean fluorescence intensity (MFI) of 184,000 produced by this BAC-transfected cell pool is significantly higher than the MFI of the plasmid-transfected pool (78,000).

These results of the pools clearly show that the probability to find stably and highly producing clones within 9 days is extremely low for conventional plasmid transfections. At the same time it indicates that there is a high probability to find a highly producing clone within 9 days after transfection with a construct containing the expression cassette with the gene of interest in a large euchromatin locus. It is also feasible that such advantageous results are found within 12 days after transfection, however, after such 12 days' time period the risk of undesired proliferation of single cell clones within the pool is higher, resulting in higher screening and characterization efforts for individual clones.

Analysis of single cells transfected with an expression cassette on either a conventional plasmid or on a BAC with a large euchromatin locus, respectively:

Table 3 shows that 2 days after transfection, BAC-transfection resulted in significantly lower fraction of eGFP-positive clones (0.5%) as compared to plasmid-eGFP transfections (2%). However, after random sorting of 96 highly producing eGFP-positive clones from each transfection one can observe a significant difference of the eGFP expression levels of the clones 21 days after transfection. Although the level of clones recovered is similar (35 out of 96 vs. 41 out of 96), the expression level of clones is significantly different: Plasmid-eGFP clones showed mostly (37 out of 41, i.e. 90%) low expression levels (MFI<6,000). Only 10% (4 out of 40) show medium levels of expression. This is well known and the reason why it is in most cases necessary to do gene amplification and prolonged cultivation for stable clones.

BAC-eGFP transfection show surprisingly a significant level (15 out of 35, i.e. 43%) of very highly producing clones (MFI>60,000) and medium producing clones (19 out of 35, i.e. 54% with MFI between 6,000 and 60,000).

Taken together, this is a surprising finding as one would have expected with the BAC-transfections a decline in expression levels after transfection similar to what is seen for the simple plasmid transfections. Due to the obviously stable and highly expressing clones in the transfections with the gene-of-interest in a euchromatin protein expression locus, it is possible to enrich with very stringent methods shortly after transfection for these clones (e.g. by antibiotic selection pressure and/or by sorting according to expression levels).

TABLE 2

| | | day 2 p.t. | day 2 p.t. | | day 9 p.t. | |
|---|---|---|---|---|---|---|
| | number of cells | % cells positive | MFI of culture | day 9 p.t. | % cells positive for | day 9 p.t. |
| aliquot 1 | transfected | for GFP | after 2 days | % viability | GFP after 9 days | MFI of culture |
| pool- | 600.000 | 0.5 | 6200 | 4 | 3.3 | 184000 |
| BAC | | | | | | |
| pool- | 600.000 | 2.0 | 6600 | 3 | 20.0 | 78000 |
| plasmid | | | | | | |

TABLE 3

| | | | day 2 p.t. | | day 21 p.t. | day 21 p.t. | day 21 p.t. |
|---|---|---|---|---|---|---|---|
| | | day 2 p.t. | cells eGFP+ | day 21 p.t. | GFP positive | GFP positive | GFP positive |
| | number of cells | % cells positive | sorted (gate | clones | clones | clones | clones |
| aliquot 2 | transfected | for eGFP | cutoff >10000) | recovered | MFI < 6000 | 6000 < MFI < 60000 | MFI > 60000 |
| clones | 600.000 | 0.5 | 96 | 35 | 1 | 19 | 15 |
| BAC | | | | | | | |
| clones | 600.000 | 2.0 | 96 | 41 | 37 | 4 | 0 |
| Plasmid | | | | | | | |

1. A method for producing a eukaryotic production cell line expressing a protein of interest (POI), comprising:
a) incorporating a gene of interest (GOI) encoding said POI into a chromosome of a eukaryotic host cell within an exogenous euchromatin protein expression locus by transfection, thereby obtaining a repertoire of recombinant host cells in a pool;
b) selecting a single cell from said pool within 12 days after transfection, wherein the selecting is at least according to the expression of said GOI or a marker indicating said expression; and
c) isolating and expanding the selected single cell, thereby obtaining the production cell line.

2. The method of claim 1, wherein said locus is integrated into the host cell via a vector comprising said locus.

3. The method of claim 2, wherein said vector is integrated randomly into the chromosome of the host cell or by site-specific integration.

4. The method of claim 1, wherein a selection marker gene is additionally incorporated into the host cell and the repertoire of recombinant host cells is maintained in said pool under corresponding selection pressure conditions, and wherein said selecting is at least according to any of the transfected marker gene, the marker, or the function of said marker.

5. The method of claim 4, wherein said selection marker gene is an antibiotic resistance marker gene or a metabolic function marker gene, and wherein said selection marker gene coexpresses a selection marker with the POI.

6. The method of claim 1, wherein method step a) comprises incorporating said GOI into said locus by site-specific integration.

7. The method of claim 1, wherein said host cell is a mammalian or avian host cell.

8. The method of claim 7, wherein the locus is a murine Rosa26 locus, or a mammalian homolog thereof.

9. The method of claim 8, wherein the host cell is a CHO cell.

10. The method of claim 1, wherein said repertoire of recombinant host cells covers host cells which differ in at least one of (i) copy number of said GOI; (ii) chromosomal locus or chromosomal loci where the GOI is incorporated; (iii) genetic stability; or (iv) epigenetic stability.

11. The method of claim 1, wherein said selecting is further according to any of cell size, cell cytoplasmic granularity, polarizability, refractive index, or cell membrane potential.

12. The method of claim 11, wherein said selecting is by a single cell sorting technique employing an optical flow cytometry method.

13. The method of claim 1, wherein said repertoire of recombinant host cells comprises at least 10,000 different clones which each differ in at least one genetic characteristic.

14. The method of claim 1, wherein the selected single cell is characterized by a GOI copy number of at least 5.

15. The method of claim 1, wherein said production cell line has a specific productivity producing the POI of at least 0.1 pcd, and wherein said production cell line is produced within less than 60 days.

16. The method of claim 1, wherein the POI is a recombinant or heterologous protein.

17. The method of claim 2, wherein the vector comprising said locus is selected from the group consisting of a bacterial artificial chromosome (BAC) vector, a P1-derived artificial chromosome (PAC), a yeast artificial chromosome (YAC), a human artificial chromosome (HAC), and a cosmid.

18. The method of claim 7, wherein said host cell is selected from the group consisting of HEK293, VERO, HeLa, Per.C6, HuNS1, U266, RPMI7932, CHO, BHK, V79, COS-7, MDCK, NIH3T3, NS0, SP2/0, or EB66 cell, and derivatives thereof.

19. The method of claim 12, wherein the single cell sorting technique is selected from the group consisting of forward light scatter (FSC), side light scatter (SSC), and selection using a microfluidic system.

20. The method of claim 16, wherein the POI is selected from the group consisting of a therapeutic protein, an immunogenic protein, a diagnostic protein, and a biocatalyst.
